(19) United States
(12) Patent Application Publication
(10) Pub. No.: US 2026/0217627 A1
(43) Pub. Date: Jul. 30, 2026

(54) Chemocatalytic Conversion of Polyaddition Polymers in Low-Value Feedstocks to Higher-Value Hydrocarbons

(71) Applicant: Aduro Clean Technologies, Inc., London (CA)

(72) Inventors: W Marcus Trygstad, Spring, TX (US); Anil K. Jhawar, London (CA); Birendra Adhikari, Sarnia (CA)

(21) Appl. No.: 19/459,759

(22) Filed: Jan. 26, 2026

Related U.S. Application Data
(60) Provisional application No. 63/750,098, filed on Jan. 27, 2025.

(51) Int. Cl. C07C 4/06; B01D 3/14; B01J 8/08

(52) U.S. Cl. CPC C07C 4/06; B01D 3/14; B01J 8/085; B01J 2208/00752

(57) ABSTRACT

Methods, systems, catalysts, and products are disclosed for converting polyaddition polymers (PAP) contained in low-value feedstocks into lower-molecular-weight hydrocarbons of higher value. In representative embodiments, a PAP-containing feedstock is combined with a first process agent comprising a mineral-based catalyst and heated under elevated temperature and pressure to promote selective carbon-carbon bond scission and formation of reactive fragments. In the presence of water, hydrogen equivalents are generated autogenously in situ by aqueous reforming of at least a portion of the reactive fragments and are used to quench other fragments, thereby forming stabilized hydrocarbon products. Conversion proceeds through iterative scission and quenching without reliance on thermolytic cracking, externally supplied hydrogen, or post-conversion hydrotreating. The methods are applicable to mixed and contaminated feedstocks comprising polyolefins, polystyrene, polyisobutylene, and tire rubber, and tolerate heteroatom-containing contaminants. The resulting products comprise substantially saturated hydrocarbons suitable for use as fuels, solvents, lubricant basestocks, or chemical feedstocks.

### CROSS-REFERENCE TO RELATED APPLICATIONS

This application claims the benefit of U.S. Provisional Patent Application Ser. No. 63/750,098, entitled Method and Apparatus for Converting Low-Value Macromolecule Feedstocks to Higher-Value Fuels and Chemicals, filed on Jan. 27, 2025, the contents of which are incorporated herein by reference in their entirety for all purposes.

### BACKGROUND

### Technical Field

This invention relates to hydrocarbon processing, and more particularly to systems and methods for efficiently producing high value products such as transportation fuels and chemical feedstocks.

### Background Information

Throughout this application, various publications, patents and published patent applications are referred to by an identifying citation. The disclosures of the publications, patents and published patent applications referenced in this application are hereby incorporated by reference into the present disclosure.

### Thermolytic Methods

Although the new art described herein applies thermal energy to enable chemical conversion of polyaddition polymers (PAP), it differs in distinct and fundamental ways from thermolytic methods applied to PAP in waste plastic streams including polystyrene (PS); and polyalkylenes, e.g., polyisobutylene (PIB) and the polyolefins (PO), which are polyethylene (PE) and polypropylene (PP). The thermolytic methods are practiced under various rubrics including pyrolysis and hydrothermal liquefaction, where the latter may be more aptly described as supercritical-water pyrolysis (SCWP) due to its application of water at temperatures and pressures well above its supercritical point and in quantities that are mass-multiples versus feedstock. Because the instant invention is not pyrolytic, it substantially avoids numerous problems inherent in the operation of thermolytic mechanisms for chemical recycling of waste plastic. The first relates to their general lack of selectivity. In what is effectively thermal cracking, thermolytic methods rely on the input of thermal energy to rupture bonds in polymer compounds. When these are saturated polyalkylenes, bonds ruptured include both carbon-carbon bonds (C—C) in the polymer skeleton and the associated carbon-hydrogen bonds (C—H). Occurrence of the latter on adjacent carbons is thermolytic dehydrogenation that yields unsaturations, e.g., carbon-carbon double bonds (C═C), which are alkene groups. The rupturing of C—C ostensibly creates reactive free radical intermediates. Numerous secondary reactions ensue, which generate products with diverse molecular weights and structure. Moreover, in what may be aptly described as carbon scrambling, the many reactions result in wholesale rearrangement of hydrocarbon functionalities native to the polymer chains and the proliferation of non-native functionalities. Thus, the product from thermolytic methods comprises compounds with a wide range of molecular weights and highly diverse structures and chemical functionality. The lack of selectivity has a further consequence of limiting possibilities to control production of both light hydrocarbon gases and heavy, carbonaceous char, which together compromise overall yields of useful hydrocarbons.

A second feature relates to the first: the rate dependency of thermolytic mechanisms and process throughput on elevated process temperatures, which commonly are at/above 440° C., makes them highly energy-intensive. Third, each occurrence of carbon-carbon bond rupturing or thermolytic dehydrogenation generates one unsaturation in product compounds. Even saturated naphthenic structures formed through carbon scrambling are understood in organic chemistry to represent unsaturations. In the hydrocarbon processing industry, alkene functionality is often problematic: it renders fuels unstable and causes coking in naphtha crackers. Although the product from SCWP is touted as having lower C═C content than that from conventional pyrolysis, which is nominally anhydrous, levels are nonetheless relatively high; products from all thermolytic methods typically must be stabilized and upgraded by post-processing, e.g., by hydrotreating.

A fourth problem is a direct consequence of the first and third: alkene functionality formed by dehydrogenation is itself an agent of functionality proliferation in the reaction mixture, as it is predisposed to form (i) allylic intermediates and (ii) conjugated dienes through further dehydrogenation. Both may undergo diverse addition reactions known to skilled artisans including ones leading to formation of aromatic functionality.

A notable, fifth issue in the application of thermolytic methods for chemical recycling of PO relates to the aforementioned scrambling of functionality that ensues after generation of free-radical intermediates. Non-polyalkylene contaminants in waste plastic feedstocks commonly include (i) polymers such as polyamides (nylons), polyesters (e.g., poly(ethylene terephthalate) esters, or PET), polyurethanes, and poly(vinylchloride), or PVC; (ii) additives that are compounded into the polyalkylenes for-use, e.g., fillers, colorants, plasticizers, and lubricants that facilitate molding or extrusion; and (iii) and miscellaneous other materials, e.g., paper. Most such contaminants contain heteroatoms, e.g., nitrogen, oxygen, and chlorine, which may become covalently integrated into product compounds through the uncontrolled side reactions involving free radicals. As with alkenes, heteroatom-containing functionalities may be substantially eliminated by post-processing techniques such as hydrotreating, which is readily available in the context of petroleum refining. Yet, such techniques do not come without cost or conditions. For example, the pyrolytic processing of polyolefinic waste plastic may be effective when integrated with petroleum refining operations but its economic relevance is constrained apart from such infrastructure. Nevertheless, the incorporation of heteroatoms from contaminants into thermolytic products represents a sixth problem with thermolytic methods applied for chemical recycling of waste plastic: the requirement to source feedstocks whose PO content typically is greater than about 95%. Although plastic sorting technologies can do this, they should be understood as producing such PO-enriched waste plastic streams at the expense of rejecting other fractions that contain lower levels of PO, e.g., <90%, and correspondingly higher levels of the aforementioned contaminants. This proportionately diminishes the total amount of PO-containing feedstocks available for chemical recycling by pyrolysis relative to total PO in the waste plastic; and it in turn increases the cost of the high-PO pyrolysis feedstocks, not only because of the low yield per tonne of waste plastic, but the additional cost incurred to dispose of the rejected lower-PO fraction.

A need therefore exists for addressing the aforementioned drawbacks of the prior art.

### SUMMARY OF THE INVENTION

The present invention relates to methods, systems, catalysts, and products for converting polyaddition polymers (PAP) contained in low value feedstocks into lower molecular weight hydrocarbons of higher value.

In one aspect, the invention provides a method for converting a PAP-containing feedstock, comprising: combining the feedstock with a first process agent (PA1) comprising a mineral based catalyst; heating the resulting mixture to conversion conditions effective to promote carbon-carbon bond scission in the PAP and to form reactive fragments; generating hydrogen equivalents autogenously in situ by interaction of said fragments with water through aqueous reforming; and quenching other reactive fragments with said hydrogen equivalents to form stabilized lower molecular weight hydrocarbons.

In another aspect, the invention provides a process method wherein PAP conversion occurs through iterative scission and quenching in at least one heated reaction zone operating under conditions of elevated temperature and pressure, wherein hydrogen equivalents required to suppress formation of unstable unsaturated products are generated within the reaction mixture without an external hydrogen supply.

In another aspect, the invention provides a system configured to convert PAP in a feedstock, the system comprising one or more reactors arranged to: (i) receive and heat the feedstock; (ii) contact the feedstock with a mineral-based catalyst to promote carbon-carbon bond scission; (iii) maintain water in the reaction mixture under conditions effective to generate hydrogen equivalents autogenously; and (iv) recover lower molecular weight hydrocarbon products.

In another aspect, the invention provides a catalyst composition suitable for promoting PAP conversion, comprising one or more naturally occurring mineral materials effective to catalyze carbon-carbon bond scission in polyaddition polymers under elevated temperature conditions while permitting in situ generation and transfer of hydrogen equivalents.

In another aspect, the invention provides a hydrocarbon product composition obtainable by the foregoing methods, comprising lower molecular weight hydrocarbons derived from PAP and characterized by substantially reduced unsaturation relative to products obtained by thermolytic decomposition of comparable feedstocks.

The invention enables efficient conversion of mixed and contaminated PAP feedstocks to valuable hydrocarbon products while avoiding reliance on thermolytic cracking, external hydrogen supply, or post conversion hydrotreating. The scope of the invention is defined by the appended claims.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF DRAWINGS

The present invention is illustrated by way of example and not limitation in the figures of the accompanying drawings, in which like references indicate similar elements and in which:

FIG. 1 is a schematic representation illustrating the formation of reactive fragments F by direct cleavage and mediated cleavage of carbon-carbon bonds in plastic derived feedstock materials.

FIG. 2 is a graphical representation showing comparative conversion results for a mixed plastic derived feedstock under selected reaction conditions, including water content, temperature, residence time, and feedstock composition.

FIG. 3 is a schematic diagram illustrating three sequential chemical reactions and corresponding expressions for concentrations of reactants and products associated with said reactions.

FIG. 4 is a graphical representation depicting relative concentration as a function of residence time for reactants and products involved in the three sequential reactions illustrated in FIG. 3.

FIG. 5 is a graphical representation showing relative net reaction rates as a function of carbon number for species A, B, C, and D.

FIG. 6 is a block flow diagram depicting selected aspects of a chemical method for conversion of plastic derived feedstock materials in accordance with one or more embodiments of the invention.

FIG. 7 illustrates generalized representations of chemical reactions associated with conversion of plastic derived feedstock materials.

FIG. 8 is a schematic representation illustrating conversion of polystyrene through benzylic carbon-carbon bond cleavage.

FIG. 9 illustrates representative undesirable reactions of reactive fragments F in the absence of quenching.

FIG. 10 is a schematic representation illustrating desulfurization during conversion of sulfur containing feedstock through carbon-sulfur bond cleavage and subsequent stabilization by hydrogen equivalents.

FIG. 11 illustrates production of hydrogen equivalents and quenching of reactive fragments through operation of a process assembly, wherein reactive hydrocarbon fragments and catalytic species are shown by way of example.

FIG. 12 is a graphical representation illustrating extent of conversion within a kinetic framework when the plastic derived feedstock comprises high density polyethylene, with representative carbon numbers and conversion factors shown for purposes of illustration.

FIG. 13 is a block flow diagram depicting selected aspects of a process method for conversion of plastic derived feedstock materials according to one or more embodiments of the invention.

### DETAILED DESCRIPTION OF THE INVENTION

In the following detailed description, reference is made to the accompanying drawings that form a part hereof, and in which is shown by way of illustration, specific embodiments in which the invention may be practiced. These embodiments are described in sufficient detail to enable those skilled in the art to practice the invention, and it is to be understood that other embodiments may be utilized. It is also to be understood that structural, procedural and system changes may be made without departing from the spirit and scope of the present invention. In addition, well-known structures, circuits and techniques have not been shown in detail in order not to obscure the understanding of this description. The following detailed description is, therefore, not to be taken in a limiting sense, and the scope of the present invention is defined by the appended claims and their equivalents.

As used in the specification and in the appended claims, the singular forms “a”, “an”, and “the” include plural referents unless the context clearly indicates otherwise. For example, reference to “an analyzer” includes a plurality of such analyzers. In another example, reference to “an analysis” includes a plurality of such analyses.

Although specific terms are employed herein, they are used in a generic and descriptive sense only and not for purposes of limitation. All terms, including technical and scientific terms, as used herein, have the same meaning as commonly understood by one of ordinary skill in the art to which this invention belongs unless a term has been otherwise defined. It will be further understood that terms, such as those defined in commonly used dictionaries, should be interpreted as having a meaning as commonly understood by a person having ordinary skill in the art to which this invention belongs. It will be further understood that terms, such as those defined in commonly used dictionaries, should be interpreted as having a meaning that is consistent with their meaning in the context of the relevant art and the present disclosure. Such commonly used terms will not be interpreted in an idealized or overly formal sense unless the disclosure herein expressly so defines otherwise.

### GENERAL OVERVIEW

This invention relates to a novel chemistry and process method for chemical recycling, which maximizes recovery of value from post-industrial and post-consumer plastic waste, and from rubber such as that in used tires, by converting the polyaddition polymers (PAP) they contain, in high yield, to low-molecular-weight hydrocarbons with high functional group purity, which are suitable as solvents, platform chemicals, lubricant basestocks, and naphtha-weight hydrocarbons, the latter being especially important as feedstock for producing olefin monomers in naphtha crackers in a circular regime. As will be shown, the instant invention is a significant and nonobvious departure from established art, providing highly improved outcomes in respect of yields, product quality, capex, and opex (the “Purpose”).

Referring now to the appended Figures, embodiments of the present invention will be more thoroughly described.

### Chemocatalytic Conversion

The inventors recognized the need for new art, given, first, the need for chemical recycling to ameliorate the global waste plastic problem and, second, the problems enumerated above that limit the capability of thermolytic methods to do so. The instant invention addresses both through chemocatalytic means novel and nonobvious. It is noted that approaches described by Trygstad et al. in commonly-assigned U.S. patent application Ser. No. 19/427,300 (hereinafter, '300), did so by combining feedstocks containing macromolecules (MM) with a First Process Agent (PA1), a Second Process Agent (PA2), and less than about 15% water relative to the feedstock to obtain a reaction mixture, where PA1 was a catalyst including certain one or more metals Mᵢ in compounds with the general formula (Mᵢ)ₐX_{b}; and PA2 was certain one or more compounds with general formula CᵤHᵥO_{w}. In examples of particular embodiments, MM such as PE, PP, and mixed polyolefins underwent chemocatalytic conversion at temperatures significantly lower than those applied in thermolytic methods, e.g., at about 360° C.-390° C., to obtain lower-molecular-weight (lower-MW) hydrocarbons containing substantially lower C—C levels compared with thermolytic methods. This was explained as the outcome of three reactions: (i) the breaking of carbon-carbon bonds (C—C) in MM backbone or chain through the operation of PA1 to obtain fragments F, which are reactive intermediates, ostensibly free radicals formed through homolytic bond scission; (ii) the in situ aqueous reforming (AR) of PA2 to generate reducing equivalents, or hydrogen equivalents H(equiv), where an equivalent is a molar equivalent; and (iii) the quenching of the intermediate fragments F by H(equiv) to form stabilized fragments F-H. The production of progressively-smaller fragments F-H by the iterative scission-quenching of the first and third reactions yielded lower-MW compounds that in particular embodiments were recovered from the product mixture by distillation.

Advantages of '300 are numerous compared with thermolytic methods, including: deconstruction of MM through the operation of PA1 to promote scission of C—C in the polymer backbone substantially selectively, in contrast with unselective thermolysis; quenching of reactive intermediates formed through C—C scission to substantially preempt secondary reactions that produce high unsaturation levels in products from thermolytic methods; the consequential suppression of reactions causing the proliferation of chemical functionalities in product compounds; the requirement for significantly lower quantities of water in the reaction mixture compared with those typically applied in SCWP, e.g., roughly 2.5% to 5% as much; lower energy intensity due to operation at lower applied temperatures; and related to the last two points, the concomitant avoidance of the thermodynamic penalty that attends generation of SCW and recovery from it of thermal energy. Yet, the instant inventors recognized that further advances may be made, to convert PAP to substantially saturated, lower-MW products, their motivation being the imperatives to improve (i) chemocatalytic art in respect of capex and opex per tonne of feedstock processed by simplifying process design; (ii) operational facility in scaled-up implementation; and (iii) conversion rates (throughput), yields, and product quality. The instant inventors have subsequently made a number of unexpected discoveries and innovations, each of which is novel and nonobvious in view of known prior art; serves to realize these imperatives; and now are described in detail.

### Inventive Distinctives

Catalyst, PA1. Initially, the inventors wished to promote the first of the three reactions enumerated above, C—C scission, using materials other than metal compounds (Mᵢ)ₐX_{b} that provide suitable catalytic activity, yet also substantial selectivity against undesirable side reactions that unacceptably compromise product quality, e.g., those causing occurrence of unsaturation, aromatization, and carbon scrambling in product compounds. An initial avenue of investigation concerned formulated catalysts, e.g., metals such as nickel and/or molybdenum deposited on substrates such as alumina or zeolites. Though highly configurable to achieve desired activity and selectivity, the instant inventors discovered that in some applications this benefit was offset by considerations including, (i) their high relative cost; (ii) susceptibility to deactivation by contaminants in feedstock and catalyzed formation of coke; (iii) deactivation by leaching of deposited metals; and (iv) moderation of activity due to chemo-physical effects associated with agglomeration and fracturing of catalyst particles. The high cost would necessitate contemplation of approaches for its recovery, reuse, and regeneration while leaching and the chemo-physical effects would limit overall catalyst longevity.

The inventors therefore reoriented their exploration toward materials, which, though having unlikely capability, compelled examination due to their simplicity, natural occurrence, abundance, and relatively low cost. They found surprisingly that, compared with compounds (Mᵢ)ₐX_{b} cited in '300, materials based on naturally-occurring minerals provided the required activity in C—C scission without compromising selectivity in relation to undesirable side reactions. The materials included ones based on: aluminosilicate compounds and aluminum silicate compounds such as montmorillonite clay, bentonite clay, kaolinite clay, and laterite clay; zeolites such as mordenite; hematite and hematite clay; and magnetite. These offered advantages over PA1 materials cited in prior art, unexpectedly delivering suitable performance while having relatively lower cost and being broadly available globally.

Controlled experiments revealed that, compared with nickel(II) nitrate hexahydrate, which is a benchmark PA1 in embodiments described in '300, the catalytic activity of minerals applied in embodiments described herein was 20%-30% lower on a same-weight basis, e.g. in PAP conversion at temperatures at/below 380° C. with water, PA2 (an H-source), and PA1 added in the amounts of 12.5%, 5%, and 2%, respectively, relative to feedstock. However, the disparity in rates of PAP conversion was not evident when performed at/above 390° C., under conditions that were otherwise the same. This finding indicated unexpectedly to the inventors that in embodiments of the instant invention that operate at/above about 390° C., mineral catalysts would not incur the performance penalty exhibited at/below 380° C., relative to nickel(II) nitrate.

Autogenous Generation of H(equiv). A second unexpected discovery relates to the generation of reducing equivalents. As described above, a feature central to embodiments of described in '300 is the quenching of reactive intermediates by H(equiv) generated in situ from Second Process Agents, PA2, and water added to the reaction mixture. Including one or more compounds with the general formula CᵤHᵥO_{w}, and referred to for convenience as an H-source, these have values of w relative to u of about 3≥u/w≥1 and include by way of nonlimiting example one or more taken from the group consisting of methanol, ethanol, ethylene glycol, glycerol, and cellulose, wherein, for the latter, v=2u−2 while for the others, v=2u+2. The production of H(equiv) by in situ AR of PA2 is facile and the quenching of reactive intermediates is substantially quantitative, owing to highly favorable energetics. Yet, on a commercial scale, practical considerations accompany the application of PA2 in embodiments of prior art, e.g., the cost to procure and store it, and the capex and opex for equipment required to convey it into the process.

Desiring to better understand the operation of PA2, the inventors performed control experiments to explore outcomes that attend variation of process temperature and the inclusion or withholding of PA2 while catalyst type and amounts of catalyst and water were fixed, relative to feedstock. With PE, PP, and mixed PE-PP feedstocks, the inventors observed that conversion to lower-MW products with low unsaturation occurred in the presence of added PA1, PA2, and water at temperatures below 390° C. in accordance with '300, where residence times were adjusted as appropriate for a given polymer. This indicates stabilization of C—C scission products F by H(equiv) generated from added PA2 and water, as described in '300. And as expected, when PA2 was withheld, products with substantially higher unsaturation levels were obtained from a given feedstock when conditions were otherwise the same. The effects of including or withholding PA2 were then examined by performing the same comparisons at various temperatures above about 390° C., where catalyst type and the amounts of catalyst and water relative to feedstock were the same as those applied in the comparisons below about 390° C. while residence times were shortened as appropriate for a given feedstock in consideration of higher reaction rates at those higher temperatures. Results obtained were unexpected: regardless of whether PA2 was included or not, unsaturation levels for a given feedstock were substantially identical at a given temperature at/above 390° C., indicating quenching of intermediates F by means other than H(equiv) generated from added H-source.

Though wishing to not be constrained by any particular theory of operation, these results may be attributed to AR of reactive intermediate fragments F. By way of illustration, PAP in the feedstock is PE and each F generated through bond scission is represented as R—CH₂CH₂, denoting a free radical in which a single electron resides in a p-orbital. If the reaction mixture includes an added H-source, then H(equiv) generated from it quantitatively quench the F at temperatures below, at, or above 390° C., provided the amounts of added water and H-source are stoichiometrically nonlimiting. But at the higher temperatures and in the absence of added H-source, quenching occurs by H(equiv) generated instead by AR of F, which by way of nonlimiting illustration is depicted in the equation, R—CH₂CH₂·+2 H₂O→CO₂+R—CH₃+5 H(equiv), where R—CH₃ is a quenched fragment F and the remaining five H(equiv) are available to quench other F. This example asserts that AR of F does not quantitatively consume F, but merely the carbon associated with the primary radical. Moreover, that equation indicates two elegant and highly beneficial features of H(equiv) production from F. First, it is limited insofar as it does not consume the entire fragment, but ostensibly only one carbon-containing moiety associated with the free radical. Second, such moieties are consumed only in quantities required to meet the stoichiometric demand for H(equiv), because the only F that can undergo AR are those not quenched due to depletion of H(equiv) generated otherwise by AR of F under the condition that quantities of available water are nonlimiting. Note that although AR of F has the same effect as AR of added PA2, e.g., the in situ generation of H(equiv) required for quenching, the term PA2 will be reserved for H-source compounds added to the process, to avoid potential confusion. The product from AR of F in particular embodiments of the art presented herein will instead be referred to as autogenous H(equiv).

The argument may be made that PA1 of the present invention or of '300 catalyzes production of H(equiv) directly by AR of saturated hydrocarbons, e.g., CₙHₙ₊₂+2n H₂O→n CO₂+ (5n+2) H(equiv). However, for the temperatures, pressures, and catalysts applied, support that this can occur appreciably is generally absent from the chemical literature. Likewise, the argument for AR of F being inherent in the art described in '300 is precluded on the grounds that substantially no F is available to undergo AR because rates are high for the energetically favored quenching reaction, e.g., R—CH₂CH₂·+H(equiv)→R—CH₂CH₃, provided that quantities of H(equiv) generated by AR of an added H-source are sufficient to meet the stoichiometric demand by all F formed through C—C scission.

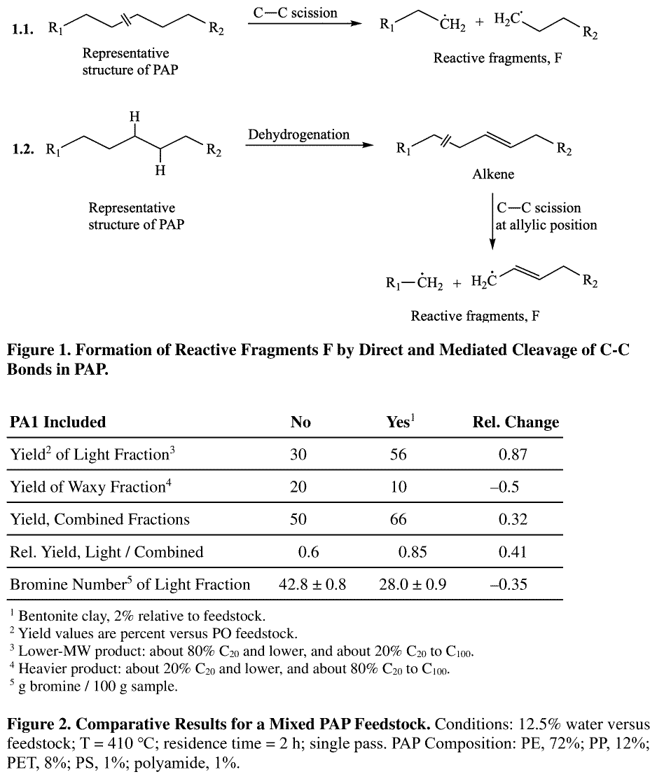

Low Unsaturation Rates at Elevated Temperatures. A third improvement not anticipated in '300 relates to the relatively low quantities of unsaturations in product compounds produced in embodiments of the instant invention. The '300 posited two mechanisms of C—C scission, as depicted in FIG. 1, both of which generate two reactive intermediates F through the operation of PA1. Direct scission generates paired F in a single step through the direct interaction of C—C in the polymer chain with metals in PA1 compounds (Mᵢ)ₐX_{b}, e.g., nickel(II) and/or cobalt(II). By contrast, a mediated mechanism ostensibly involves two steps: initial dehydrogenation through the catalytic operation of PA1 to break C—H bonds on adjacent carbon atoms in the polymer, producing an alkene; and subsequent C—C scission in the latter at the allylic position to produce two free radicals, e.g., an alkyl radical and a more stable allylic radical. The direct mechanism was described as desirable because the rapid, substantially quantitative quenching of the F yields two fragments F-H and preempts secondary reactions that can lead to production of unsaturated compounds. By contrast, the mediated mechanism was deemed undesirable, as unsaturations formed through initial dehydrogenation step persist in product compounds even after quantitative quenching of F. Having observed that unsaturation levels in the lower-MW products increase with temperature, those applied in examples presented in '300 were at or below about 390° C. to minimize this effect; temperatures there were selected in respect of the susceptibility for different PAP to chemocatalytic deconstruction, e.g., 390° C. for PE, 370° C. for PP, and 360° C. for PS. The additional motivation for limiting the temperature in '300 relates to the possibility that the catalyzed dehydrogenation is not necessarily followed by catalyzed C—C scission described for the mediated mechanism, in which case the alkene functionality also persists in product compounds. Accordingly, temperatures applied in '300 achieved reasonable overall conversion rates while minimizing unsaturation levels in the product.

However, the inventors hereof found that polyolefin polymers subjected to chemocatalytic conversion in particular embodiments yielded unexpectedly low unsaturation levels in the lower-MW products at temperatures greater than 390° C. Measured as bromine number (BN, g Br₂/100 g sample), unsaturations were observed to increase with temperature. Yet, as shown in FIG. 2, the BN of 42.8 for the light product fraction produced under thermolytic conditions was over 50% higher than the 28.0 value under chemocatalytic conditions (same residence time, temperature, and water). This was a departure from the expectation that thermolytic effects would become predominant above about 400° C., especially thermolytic dehydrogenation. The data also indicate that quenching of reactive intermediate fragments F occurs rapidly upon catalyzed C—C scission, suppressing subsequent reactions that generate unsaturations in products, as occurs unchecked in pyrolysis.

Summary Embodiments of the present invention are highly cost-advantaged compared with '300 because it applies inexpensive mineral-based materials as PA1 and achieves quenching by in situ generation of H(equiv) without the requirement for addition of an H-source, PA2, as taught in '300. Further, it is highly cost-advantaged compared with pyrolysis methods insofar as it operates at lower temperatures and avoids the thermodynamic penalty of SCWP while producing lower-MW products with substantially lower unsaturation levels.

Terminology: Conversion versus Decomposition. In view of the inventive distinctives enumerated so far, the inventors recognized the possibility for thermolytic mechanisms to operate at temperatures applied in embodiments of the present invention. Investigations confirmed that PAP was indeed altered physically and chemically when PA1 was withheld from the reaction mixture. Yet, non-catalyzed and catalyzed products obtained from pure PAP (no contaminants) differed in respect of both quantity and quality when subjected to otherwise equivalent single-pass conditions. Results representative of those differences are given in FIG. 2 where the feedstock instead was waste plastic.

Given that those results were obtained under single-pass conditions in a closed batch reactor, they should be regarded as indicative and nonlimiting. Indeed, particular embodiments will be described that enhance conversion rates and total yields of the lower-MW, lighter product fraction beyond those depicted in FIG. 2. Yet the data show clearly, first, that at modest temperatures applied in particular embodiments of the present invention, e.g., 410° C., chemocatalytic rates are markedly higher than thermolytic rates, which is attributable to catalysis by PA1. Second, the total yield is also higher, as is the amount of the highly-valued light fraction relative to that total. And third, as previously discussed, unsaturation levels of that fraction are substantially lower. The operation of different mechanisms that provide differing outcomes demands application of different terms: chemocatalytic conversion (“conversion”); and thermolytic decomposition (“pyrolysis” in all its forms). Although the latter effects a type of transformation, conversion is reserved presently for the substantially selective operation of chemocatalysis in embodiments described herein; the unselective means of pyrolysis methods for producing lower-MW products from PAP are more aptly described not as conversion but thermolytic decomposition.

Novel Kinetic Framework. The chemocatalytic art taught in '300 describes the combining of MM-containing waste plastic feedstocks with a first process agent (PA1, catalyst), a second process agent (PA2, H-source), and water in process apparatus configured to receive, heat, and mutually disperse the same to obtain a reaction mixture, which then is maintained at temperatures of about 360 to 390° C. and pressures of about 200 to 1500 psi for a residence time suitable to generate a product mixture containing lower-MW compounds that are substantially saturated. It further acknowledges that because products from single-pass deconstruction of MM span a wide carbon number range, e.g. from single digits into the hundreds, particular embodiments beneficially maximize yields of desired lower-MW compounds, e.g., those with carbon numbers below about 32 and below about 20, through continuous-flow apparatus configured to provide multiple-pass deconstruction of higher-carbon-number compounds.

Yet, the inventors hereof recognized that a process paradox resides at the heart of such an approach: the simultaneous requirements to (i) deconstruct MM in feedstocks and the high-MW fragments F-H initially obtained from them, whose carbon numbers are very high, e.g., from perhaps a thousand to tens of thousands; and (ii) generate the desired lower-MW compounds in high yield; and (iii) do so without incurring net yield losses in liquid yields through overconversion that generates large quantities of low-MW byproduct gases. Multi-pass strategies may be applied to address these contradictory demands, yet achievable throughput is limited by the differential conversion rates of high-, medium-, and low-carbon-number PAP and F-H in the reaction mixture. Not wishing to be constrained by any particular theory of operation, those rates relate principally to the MW-dependency of diffusivity, e.g., D□M_{w}⁻ⁿ, where typically n<1 for lower-MW compounds but n>1 for polymers in a medium of defined viscosity. Together with concentration, this governs the rate at which PAP or F-H of the different MW adsorb onto PA1, e.g., the number of interactions per unit time in the reaction mixture, e.g., s⁻¹ (moles/L)⁻¹. Given this physico-chemical reality, the inventors sought to identify a strategy for configuring embodiments that solve the paradox.

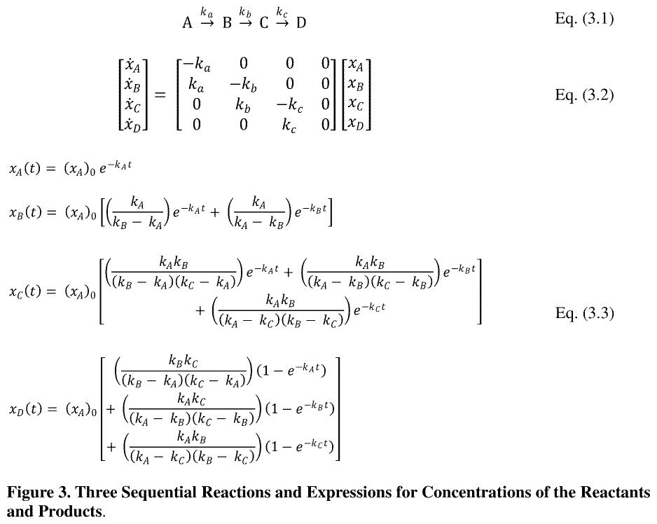

First, they recognized the nonobvious possibility to reappropriate the treatment in the chemical literature of kinetics for sequential reactions in which a starting reactant A is converted to a final product D via intermediates B and C. FIG. 3 depicts this in Equation 3.1, where kₐ, k_{b}, and k_{c}>0; each is a distinct rate constant corresponding to each of three different, unimolecular chemical reactions; each reaction in the cascade has mass action kinetics, e.g., the rate of each chemical reaction is proportional to the product of the concentrations of the reacting chemical species; and each rate constant includes a concentration factor for catalyst concentration, which is constant across the three reactions.

The conversion of PAP may be regarded as also occurring via cascading reactions, except now the reactant A is PAP in the feedstock while B, C, and D comprise subsets of successively smaller polymer fragments with D being the lower-MW product. But this situation differs in two distinct ways from the classical treatment of three sequential reactions. First, instead of occurring via three different reactions, the scission of a bond between two adjacent carbon atoms in the polymer backbone may be regarded as identical in all three steps. Though wishing to not be bound by any particular theory of operation, the rate constant for scission events catalyzed by the operation of PA1 on C—C is expected to be substantially the same in A, B, and C, e.g., it is substantially independent of the length of substituent chains extending from a given C—C. Yet, MW-dependent mass transfer rates would be expected to govern the adsorption onto PA1 of compounds in each of those subsets and/or interaction of C—C in the same with active sites in PA1. Thus, while the rate constant for C—C scission does not change with the MW of A, B, or C, the respective net rate constants for the reaction of each (Equation 3.1) would be expected to increase, e.g., kₐ<k_{b}<k_{c}.

A second implication is that B, C, and D do not comprise discrete chemical compounds but populations of F-H compounds wherein the carbon number of each has a distribution characterized by a successively lower average and range, just as polymers A in feedstocks except those that are crosslinked, which in general are indeterminately large. Thus, B comprises a population of fragments (F-H)_{B} derived the parent PAP, which is A, and whose average carbon number is lower than that of the PAP. Similarly, C is (F-H)_{C} and D is (F-H)_{D}, where each has a lower average carbon number than its corresponding precursor according to Equation 3.1; and where D is the final, desired lower-MW product obtained by conversion of PAP. Likewise, kₐ, k_{b}, and k_{c} represent the overall rate constants for scission-quenching of the populations A, B, and C, e.g., each is the aggregate of rates for adsorption onto PA1, interaction of C—C with active sites in PA1, and C—C scission.

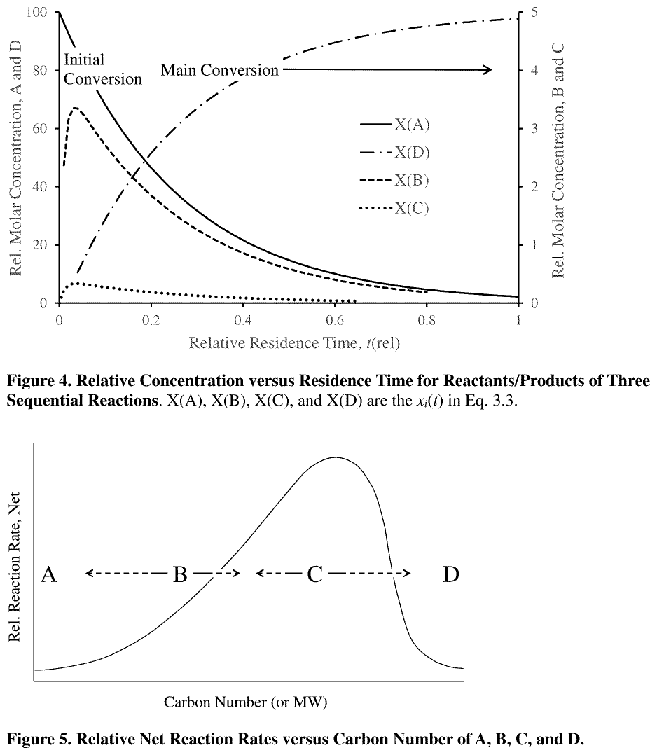

Skilled artisans will appreciate five aspects of the kinetic framework illustrated in FIG. 3. First, it is a nonlimiting illustration that assumes the parent PAP has some certain average carbon number and an associated carbon number distribution. Although the kinetic framework therefore does not apply directly to crosslinked PAP, understandings from it do, as regards the operation of embodiments described herein. Second, the significance of B and C is not that they denote certain, defined populations of compounds; rather, their significance resides in their operation as constructs that give insight into the progressive conversion of PAP to lower-MW compounds D, which in turn has implications for configuring embodiments that optimize PAP conversion. Third, Equation 3.1 does not imply that A reacts quantitatively to form B before B starts reacting to form C, or that production of D awaits the quantitative production or C from B. Rather, FIG. 4 shows that the products B, C, and D form concurrently. A salient fourth point is that the relative concentrations plotted in FIG. 4 are mole-basis, as is customary in connection with kinetics. Given that the molecular weights decrease in the progression from A to B to C to D, weight-percent values for B, C, and D, relative to that for A, would be much lower than the corresponding mole-percent values at all t(rel)≥about 0.1.

The chemical literature covers well the equations governing the kinetics of two different sequential reactions; the coverage for three is scant, possibly due to the complexity evident in Equations 3.2 and 3.3; and missing altogether is the treatment when the same reaction yields three products of differing MW, as contemplated here. Equation 3.2 presents a system of linear ordinary differential equations while Equation 3.3 presents the result from their integration, x (t), which are the concentrations of the i^{th} subset population at the time t. FIG. 4 presents by way of non-limiting illustration the relative concentrations of A, B, C, and D provided by Equation 3.3, across a normalized timescale of 0 to 1.0. Cursory inspection reveals that, (i) shortly after PAP begins to undergo conversion, the molar concentrations of B and C relative to A appear to be constant, e.g., the ratios of their concentrations do not change for t(rel) from about 0.1 to 1.0; (ii) the molar concentration of B is low relative to A, owing to its higher mobility, and that of C is lower still due to its even higher mobility; (iii) at relative residence times of about 0.1 and 0.2, about 65 and 50 mole-percent of A, respectively, has been converted to B; and (iv) the yield of the lower-MW product D is about 85% after a relative residence time of 0.6.

Implications and a Further Inventive Distinctive. The third and fourth preceding points indicate strikingly that the rates for consumption of A largely determine overall conversion rates. This may be understood as the consequence of two effects. Early in conversion, the reactant present in highest concentration is A and most of the catalyst is engaged in the first reaction, A→B. But when the relative residence time is around 0.1, molar concentrations of B and C reach their maximum relative to A, after which point all three reactants compete for active sites in the catalyst while the ability of A to do so is constrained for reasons described. Those same reasons remind of previously-identified concerns about overconversion, e.g. lowered yields of liquid products due to ongoing scission-quenching of D to produce very-low-MW hydrocarbons of limited value, e.g., methane, ethane, propane, and butanes. The inventors expected that (F-H)_{D} would exhibit accelerated scission-quenching due to its high mobility compared with A, B, and C. And they were aware that thermolytic (cracking) mechanisms in pyrolysis methods operate without regard for molecular weight of components in the incipient pyrolysis oil product. However, the inventors observed unexpectedly in the operation of particular embodiments hereof that reaction of compounds D did not continue unabated but rather exhibited a lower propensity to undergo further scission-quenching. They then reasoned that instead of being contradictory, this experimental outcome was consistent with the mobility of compounds D being so high, relative to that of A, B, and C, as to substantially diminish (F-H)_{D} adsorption onto PA1 and more so the interaction of its C—C catalytic sites on PA1. This does not altogether preclude but substantially mitigates undesirable production of very-low-MW compounds from D. And the extent to which it does represents a self-limiting function, which is non-obvious but highly beneficial, and whose operation is substantially lacking in thermolytic art.

FIG. 5 provides a non-limiting depiction of a feature related to the kinetic framework for embodiments of the present invention, as described hereinabove. The curve indicates how the net rate constants for scission-quenching vary as a function of carbon numbers spanned by A, B, C, and D. Though qualitative, the curve conveys important quantitative realities: the relative scission-quenching rates for Initial Conversion of A are relatively low; those for B are higher; those for C are higher still; and those of D are low. The inventors used this understanding about the lower reaction rates for A and D compared with B and C to inform the configuration of exemplary embodiments, which are directed toward the aforementioned imperatives and will be detailed hereinbelow.

Summary The inventive distinctives of embodiments of art described herein relate to at least four unexpected discoveries through investigations by the inventors seeking increased econo-commercial performance in scaleup compared with possibilities provided with other approaches. First, certain minerals were found to be efficacious as First Process Agents (PA1) for catalyzing C—C scission in certain PAP while also having broad availability and relatively low cost. Second, discovery that in situ generation of H(equiv) occurs at temperatures above about 390° C., without the requirement taught in '300 to add a Second Process Agent (PA2), obviates costs related to material procurement, capital equipment, and its operation. Third, evaluation of certain PA1 minerals revealed that their activity in C—C bond scission is not accompanied by disproportionately higher rates of catalyzed dehydrogenation, permitting application of higher temperatures that increase conversion rates without incurring excessive unsaturation levels in lower-MW products. Fourth, the trend toward increasing scission-quenching rates that generally attends the progressive reduction in carbon numbers of F-H reverses in the lower-MW limit to suppress yield losses by overconversion. Additionally, the inventors' nonobvious appropriation of kinetics for three sequential reactions provides insights novel and nonobvious, which are applied in exemplary embodiments to achieve improved conversion efficiency in support of the stated Purpose of the present invention. Having established those novel, nonobvious, and practical distinctives versus prior art, aspects of the present invention will now be described in greater detail.

### Chemical Method

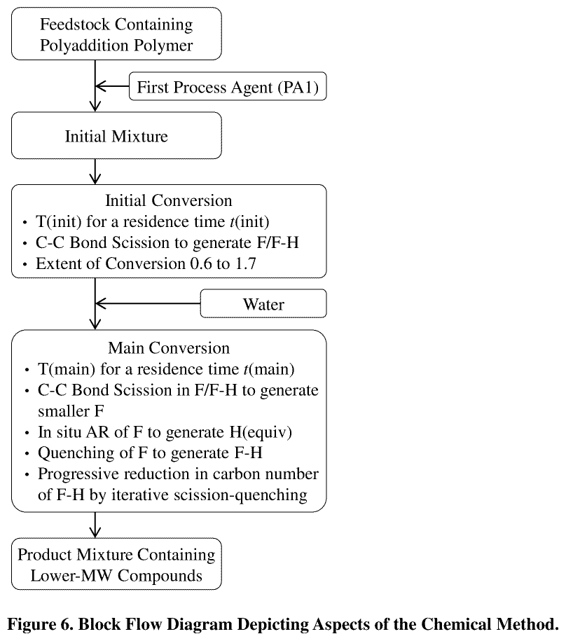

Referring now to FIG. 6, particular embodiments are configured to realize the aforementioned distinctives for converting polyaddition polymers (PAP) in lower-value feedstocks to higher-value products including lower-MW compounds, where:
(a) Feedstocks include waste plastics and tire rubber; and
(b) The PAP in waste plastics include polystyrene (PS) and polyalkylenes, where the latter includes polyisobutylene (PIB) and polyolefins (PO), which are (PE) and polypropylene (PP); and those in tire rubber are co-polymers synthesized with natural rubber and synthetic prepolymers derived from petroleum including styrene, butadiene, isoprene, ethylene, and propylene;
(c) Conversion occurs by agency of a First Process Agent, PA1, combined with the feedstock to obtain an initial mixture;
(d) PA1 is a catalyst that promotes the scission of carbon-carbon bonds (C—C) in the PAP backbone or chains upon the heating of the initial mixture to process temperatures of T(init) and contacting of the PAP with the PA1, thereby obtaining an initial reaction mixture;
(e) Initial conversion occurs in the initial reaction mixture at T(init) applied for residence times t(init), which promote C—C scission that generates, from the PAP, intermediate fragments F whose average size is between about one-fourth and one-fiftieth that of the PAP in the feedstock, wherein size relates to carbon number or molecular weight;
(f) Water in the feedstock and/or PA1, and optionally water added to and dispersed in the initial reaction mixture, combines with F in self-regulating aqueous reforming (AR) to generate hydrogen equivalents, H(equiv), that effect in situ quenching of other F to obtain stabilized fragments F-H;
(g) Main conversion occurs through (i) subsequent, iterative C—C scission in F and/or F—H from Initial Conversion, by agency of PA1, and (ii) quenching of F by autogenous H(equiv) and (iii) the addition of water in quantities sufficient to satisfy the stoichiometric demand in the self-limiting AR of F that generates H(equiv), and (iv) the application of temperatures T(main) applied for residence times t(main), all of which obtain a main reaction mixture in which the iterative scission-quenching produces progressively smaller, stabilized fragments F-H;
(h) Main conversion continues until a product mixture is obtained, which includes F-H with the qualities desired of the lower-MW compounds, which qualities relate to average molecular weight or average carbon number.

Aspects of the present invention are now described, which relate to the operation of the chemical method.

PAP in Feedstocks. Embodiments of the invention described herein operate on PAP contained in feedstocks, wherein the polymer backbone, chains, matrix, or network consist substantially of contiguous carbon atoms assembled by the chain reaction of unsaturated functional groups in monomers, e.g., by free radical or ionic mechanisms. The chains in certain PAP are substantially linear chains, whereas in certain other PAP the chains contain branching and in certain other cases they are crossed-linked in a three-dimensional matrix or network. Examples of these based on polyethylene (PE) include, respectively, high-density PE (HDPE); linear low-density PE (LLDPE) and low-density PE (LDPE); and crosslinked PE (PEX). Other nonlimiting examples of PAP include polypropylene, or PP; polystyrene, or PS; and poly(isobutylene), or PIB; and the hydrocarbon polymer fraction of tire rubber, or TR, which comprises a highly-crosslinked matrix. The designations PP, PS, and PIB connote homopolymers, yet numerous commercial variations exist on these PAP, including random and block copolymers synthesized from a plurality of monomers. Thus, these designations are nonlimiting, being indicative polymers having been synthesized with but not necessarily exclusively from their namesake monomers.

Mechanisms of PAP Conversion. The chemical method produces lower-MW product compounds through the operation of a First Process Agent, PA1, which is a catalyst that promotes C—C scission in the backbone or chains of PAP. Though wanting to not be bound by any particular theory of operation, the activity of PA1 dispersed in the reaction mixture may be explained in terms of interaction between C—C in polymer chains with active sites in the one or more minerals, mineral crystals, or mineral phases, where that interaction lowers the activation energy for production of incipient reactive fragments F by mechanisms known to skilled artisans, e.g., Frontier Molecular Orbital theory whereby highest-occupied molecular orbitals (HOMO) or lowest-unoccupied molecular orbitals (LUMO) of C—C interact with, respectively, unoccupied or occupied orbitals associated with atoms in the mineral-based compounds of PA1. At temperatures applied in particular embodiments, those compounds provide the required activity and selectivity in C—C scission, yet substantially without also promoting previously-identified undesirable side reactions to extents that unacceptably compromise product quality.

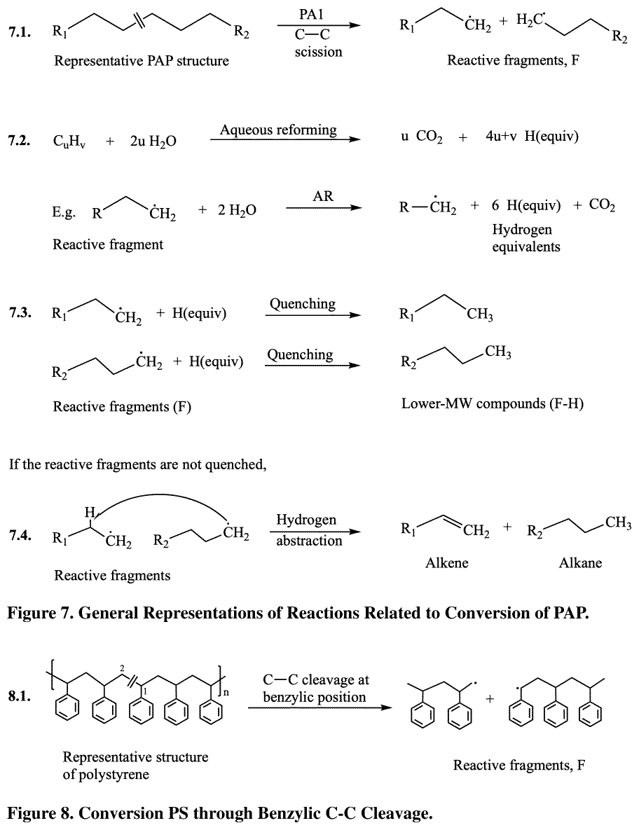

FIG. 7 presents generalized depictions of reactions involved in conversion of PAP to lower-MW compounds through the chemical method of the present invention, wherein bond scission occurs in a fashion that may be regarded as direct or mediated, as described previously (FIG. 1). When the PAP contain aromatic functionality appended to an alkyl chain, such as in styrenic moieties of PS or TR, direct C—C bond scission occurs particularly readily in the alkyl chain at C₁-C₂ position relative to the aromatic group (FIG. 8). The reason, well-known to those skilled in the art, is that one of two free radicals formed is benzyloid, which is highly stabilized due to delocalization of the free electron into the aromatic system.

Three cases have been identified: Case 1 is direct scission of C—C bonds in a moiety consisting of saturated hydrocarbon chains containing branching or not (Reaction 1.1); Case 2 is bond scission that occurs through intermediate agency of alkene functionality formed by dehydrogenation across adjacent carbons (Reaction 1.2); and Case 3 is direct scission at the C₁-C₂ position of an alkyl substituent on an aromatic group (Reaction 8.1). In embodiments that apply a particular PA1 at a particular temperature to promote PAP conversion, rates of bond scission will vary as Case 1<Case 2<Case 3, which is a consequence of factors commonly understood by skilled artisans.

Given the objective of avoiding elevated unsaturation associated with thermolytic methods, Case 2 is undesirable because alkene equivalents, formed through dehydrogenation, substantially persist even after the operation of H(equiv) in particular embodiments to quench reactive intermediates from bond scission. Known well to skilled artisans is the fact that saturation of alkene functionalities typically occurs under severe conditions, e.g., molecular hydrogen applied at elevated temperature and pressures in the presence of noble-metal catalysts. Absent such conditions in examples described in '300, it substantially minimized unsaturation by applying temperatures below about 390° C. where rates of dehydrogenation occurring by agency of the PA1 employed were known to be relatively low. Notably, the inventors of the present art confirmed in control studies that H(equiv) generated in embodiments of the present invention cause substantially no reduction of alkene functionality in aliphatic compounds.

PA1 Composition. The PA1 applied in '300 includes one or more discrete transition metal compounds with the general formula (Mᵢ)ₐX_{b}, wherein one or more metals Mᵢ are the ostensible agents of bond scission. In embodiments of the present invention, PA1 is instead one or more materials based on naturally-occurring minerals, e.g., naturally occurring geological materials comprising one or more inorganic compounds, wherein (i) compositions are defined or variable; (ii) crystal or phase structures are defined or variable; and, when including a plurality of compounds, (iii) they may exist within a common crystal framework or mineral phase, and/or (iv) each may exist in a distinct crystal framework or mineral phase, and/or (v) one or more compounds may exist within the crystal framework or phase of another. Nonlimiting examples of such minerals include: aluminosilicate compounds and aluminum silicate compounds such as montmorillonite clay, bentonite clay, kaolinite clay, and laterite clay; mordenite; hematite and hematite clay; and magnetite. Compared with other minerals, e.g., synthetic zeolites, those materials were found to promote the desirable C—C scission in PAP to relatively greater extents than the aforementioned three undesirable reactions.

Quenching of Reactive Intermediates. Reaction 7.1 depicts PAP conversion by C—C scission through the operation of PA1 to yield unstable, reactive intermediates that ostensibly are free radicals, which, if not quenched in situ, go on to yield one unsaturation for each C—C broken (depicted by Reaction 7.4). Having established the unsuitability of molecular hydrogen for in situ quenching, '300 describes a novel, nonobvious method that achieves the desired quenching by reducing equivalents, H(equiv), generated in situ by aqueous reforming (AR), at applied temperatures of less than about 390° C., of a Second Process Agent (PA2) added to the reaction mixture. In the present invention, the chemical method achieves quenching without the requirement for added PA2, the required H(equiv) instead being generated autogenously. Despite PA2 applied in '300 being generally less expensive and more facile than product post-processing by catalytic hydrogenation, the present invention altogether avoids costs associated with both approaches, thereby reducing capex and opex.

Benefits of the Chemical Method. Preceding discussion establishes that (i) the objective of any means for PAP conversion contemplated in the present invention is to obtain useful, lower-MW compounds of higher value; (ii) conversion necessarily depends on breaking C—C bonds that define PAP structure, e.g., its backbone, chain, matrix, or network, to obtain lower-MW fragments; (iii) whether a C—C is broken by means of thermolytic or catalytic, the result is one unsaturation in every two fragments produced, if not performed in a reducing environment; and when not, (iv) high unsaturation levels in lower-MW products from PAP conversion limits their value and usefulness; (v) thermolytic methods further cause undesirable integration of heteroatoms into the covalent structure of product hydrocarbons, e.g., N and O found in contaminants of waste plastics that contaminate feedstocks; and (vi) the relevance of traditional transformation of PAP by thermolytic methods is therefore conditioned on supplementation with costly product post-processing to achieve unsaturation acceptable levels and, as appropriate, lower levels of N and O, where such post-processing typically is catalytic application of molecular hydrogen, e.g., hydrotreating.

As regards (iii), a reducing environment is one in which reducing equivalents are available in the reaction mixture to quench F, where reducing equivalents are hydrogen equivalents; and the two terms are synonymous for present purposes, both denoting moles of elemental hydrogen (H atoms) irrespective of their location or chemical nature. Embodiments of the invention described herein substantially avoid the enumerated issues (iii) to (vi) through the operation of autogenous H(equiv) produced by in situ AR of F, provided two conditions are satisfied: amounts of water present in the reaction are sufficient to ensure that amounts of H(equiv) produced are stoichiometrically nonlimiting; and the process method ensures that the reducing equivalents substantially pervade the reaction mixture. Because alkyl free radical species, such as those thought to form in catalyzed bond scission in particular embodiments, are well known to be highly reactive, quenching by Reaction 7.3 is highly energetic, i.e. AG is highly negative. This ensures that quenching rates are likewise very high. Though wishing not to be bound by any particular theory of operation, those high rates ensure that quenching of reactive intermediates from catalyzed bond scission is substantially quantitative and consequently preempts slower hydrogen transfer reactions that lead to unsaturations in fragments generated by conversion of PAP.

To appreciate the efficacy of particular embodiments for minimizing unsaturation in lower-MW products, suppose that no quenching attended the breaking of C—C in PE, whether by catalyzed C—C scission or by thermolytic cracking, and that each bond broken produced one carbon-carbon double bond. Then, the BN would be about 75 for the lower-MW product whose average carbon number is about 15. For particular embodiments described herein, BN ranges from about 17 to 41, depending on severity. The midpoint in that range, 29, compares favorably with the value of 28 presented in FIG. 2 for a mixed feedstock containing PE and PP. The perspective provided by BN values for pyrolysis oil is quite different. Although BN values for pyrolysis products vary widely according to the process and the pyrolysis oil fraction analyzed, analysis of representative samples indicated that values 80 to 110 are likewise representative, which is about 2.9 to 3.9 times greater than the 28-29 value for embodiments of the present invention. Referring once again to FIG. 2, consider that BN for the product obtained without catalyst, under conditions that were otherwise identical, was 50% greater than with added PA1 when the applied temperature was 410° C. Given that pyrolysis methods commonly operate at 450 to 500° C., the values 80 to 110 are highly credible and underscore benefits of embodiments that effect PAP conversion by the chemical method.

Beyond the benefits of quenching, water beneficially participates in AR of incipient coke that happens to form in the reaction mixture. In hydrocarbon PAP compounds with the formula CₓH_{y}, the H/C mole ratio y/x has a value≥1, e.g., 2, 2, and 1 in PE, PP, and PS, respectively. For incipient coke, y/x<1 whereas in fully formed coke consists substantially of elemental carbon, e.g., y<<x. Under process conditions in embodiments of the present invention, coke undergoes AR according to Equation 7.2 to produce CO₂ and H(equiv).

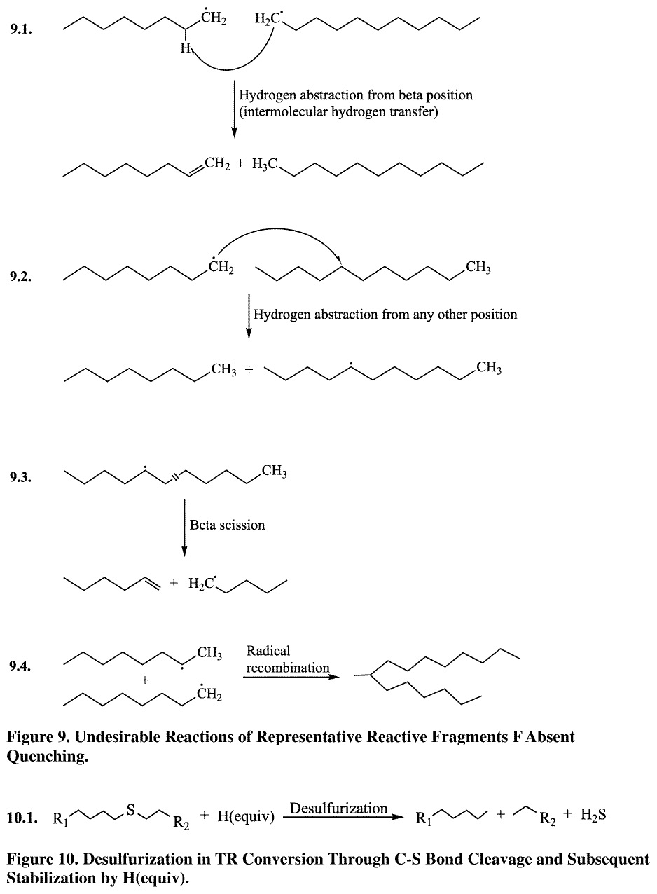

Benefits of Autogenous H(equiv) and Quenching. Beyond suppressing unsaturation in products of PAP conversion and formation of coke, the effectiveness of H(equiv) for quenching reactive intermediates in embodiments of the present invention bestows three significant benefits over thermolytic methods. First, the selfsame quenching substantially suppresses other reactions of free radicals, e.g., inter- and intramolecular hydrogen transfer, beta scission, radical recombination reactions that produce unsaturations and branching according to reactions shown in FIG. 9. Consequently, structures of PAP conversion products relate substantially recognizably to those of the parent PAP. For example, embodiments of the present invention obtain product compounds from PE consisting substantially of straight-chain alkanes with single-digit percentages by weight of alkene and aromatic functionalities and substantially no branching. Similarly, products from PP are substantially quaque altera carbonis methylated alkanes (or more conveniently, methyloligopropylene compounds, MOP), which are straight-chain alkanes bearing a methyl group on every second carbon in the molecular backbone, also with low alkene and aromatic functionality. Compounds obtained from PE or PP in particular embodiments of the present invention are, respectively, paraffins and MOP suitable as lube stocks, solvents, and waxes; and those from PS include mono-substituted benzene compounds such as toluene and ethyl benzene, and di- and tri-phenyl alkanes. All are commercially relevant as value-added solvents and feedstocks.

This contrasts with the carbon scrambling that is characteristically rampant in pyrolysis methods that rely on cracking. The absence of in situ quenching permits: diverse reactions of alkene functionalities (unsaturations) formed through that cracking; the uncontrolled, wholesale rearrangement of hydrocarbon structures in cracking products; and the consequential proliferation of hydrocarbon functionality. This generates a complex mixture of compounds bearing only remote resemblance to PAP structures, if any, and significantly impairs their path to commercial applications.

Second, stabilization of intermediates from C—C bond scission substantially precludes covalent integration of heteroatoms from feedstock contaminants into the lower-MW products of PAP conversion. Considering the realm of plastic recycling, mature mechanical sorting technologies are capable of generating plastic streams that are enriched in a particular type of plastic while going far to minimize contamination by paper and other types of plastics. Such sorting may produce waste polyolefin streams with contaminants at levels of 5% to 10%. Even then, the enriched polyolefin stream may contain low percentages of N and O, which reside in residual cellulose; in poly(ethylene terephthalate ester) or PET; and in nylon. Because thermolytic methods operate indiscriminately on bulk and trace components alike, N and O become covalently integrated into hydrocarbon products from polyolefin pyrolysis, through the aforementioned scrambling. Quenching in embodiments of the present invention substantially avoids this problem; the heteroatoms therefore are retained in derivatives of cellulose, nylon, and PET instead of being incorporated covalently into the structures of the desired hydrocarbon products. This attests to the operation of embodiments of the instant invention that is highly tolerant of feedstock contaminants.

Third, as regards heteroatoms when the feedstock is TR, the effect of the operation of PA1 and H(equiv) in particular embodiments relates somewhat oppositely to that described for waste polyolefin plastic feedstocks. In the latter, they suppress or prevent covalent integration of heteroatoms from contaminants into the low-MW products. In TR, a heteroatom is already integrated in the feedstock: sulfur exists at low-percentage levels as covalently-bonded mono-, di-, or polysulfidic linkages between carbon atoms. Wanting again to not be limited by any particular theory of operation, embodiments of the instant invention apply H(equiv) to reductively liberate sulfur as H₂S in analogy with hydrodesulfurization (FIG. 10). This is effectively the opposite of heteroatom integration into products from thermolytic methods; the chemocatalytic method instead removes the heteroatom.

A further benefit of particular embodiments presented herein is the robustness of their operation toward contaminants in feedstocks. As already described, quenching by H(equiv) substantially suppresses scrambling associated with thermolytic methods such that compounds produced substantially retain the purity inherent in PAP of feedstocks, e.g., the chemical functionality and elemental composition are substantially preserved from insinuation by heteroatoms in contaminants. The same holds for PA1 compounds: due to their structural and chemical simplicity, they substantially resist being compromised by feedstock contaminants in the reaction mixture, e.g., their catalytic activity remains robust.

Stoichiometric Demand for H(equiv) and Water. As indicated hereinabove, particular embodiments generate H(equiv) autogenously in amounts required to ensure quantitative quenching of reactive intermediates formed by catalytic bond scission in PAP. Those amounts may be estimated from knowledge about the number of bond scission equivalents (events) per kg PAP in the feedstock required to achieve the extent of PAP conversion corresponding to the desired MW of conversion products. This is straightforward in the case of feedstocks containing polyaddition polymers like PE, PP, and PS; the demand for H(equiv)/kg PAP may be calculated as the moles of hydrocarbon compounds/kg product produced. For example, if the average carbon number for those compounds is 18 C/molecule, the demand for reducing equivalents is about 8 H(equiv)/kg PAP. Whereas '300 is predicated on supplying amounts of PA2 suitable to generate H(equiv) in quantities sufficient to quench F generated through C—C scission, the production of H(equiv) in embodiments of the instant invention is self-regulating, e.g., quantities of F consumed through in situ AR are only those required to quench F produced.

Although the corresponding amount of water is about 4% relative to PAP in the feedstock, additional water typically is required to also to satisfy the demand for water consumed in hydrolysis of contaminant polymers like polyesters and polyamides, e.g., PET and nylon. For example, contaminant PET readily hydrolyzes to liberate the monomers terephthalic acid and ethylene glycol (EG); and the latter undergoes AR to generate H(equiv).

The operation of particular embodiments benefits from excess water: its high heat capacity and mobility enhance heat- and mass transfer in the reaction mixture; and it is a convenient vehicle for achieving elevated pressures that promote desirable reactions and suppress undesirable ones, where the former include AR and bond scission, whose rates are higher at pressures applied in particular embodiments, and where undesirable reactions include coke formation, which may impact catalyst activity. Accordingly, particular embodiments operate with amounts of water from all sources totaling about 10-15% relative to feedstock, including any moisture in feedstocks; water associated with added PA1 compounds; and water added to the reactor. Pressure in the reactor increases autogenously due to the vapor pressure of that water and of lower-MW products of PAP conversion and to byproduct CO₂ and H₂S.

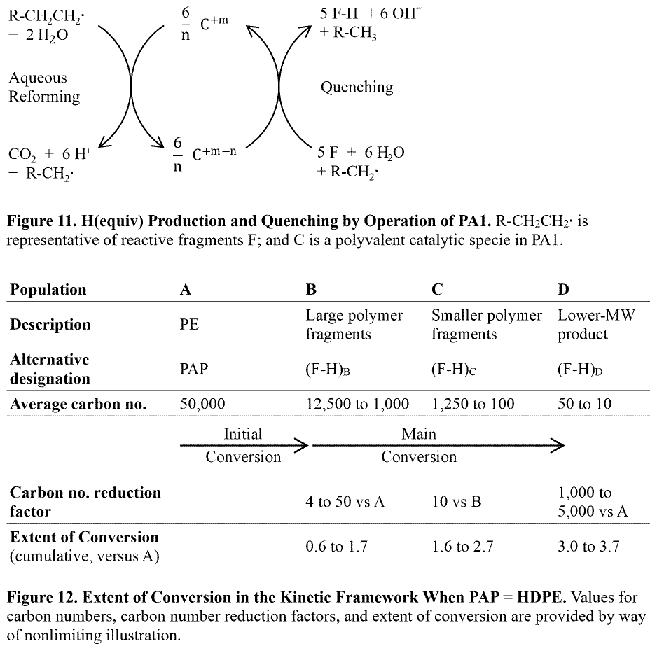

Role of PA1 in Quenching of F. The accounting in extant art is very limited concerning possible mechanisms by which PA2 compounds applied in '300 generate molecular hydrogen, much less for operative mechanisms that generate H(equiv) in embodiments of the present invention. Though desiring to not be constrained by any particular theory of operation, the inventors envisioned a twofold catalytic role for PA1: promote C—C scission in PAP; and facilitate transfer of reducing equivalents from F to reactive intermediates generated by that scission according to the scheme presented in FIG. 11. In this scheme, one or more polyvalent catalytic species C in PA1 have a plurality of available oxidation states, e.g., C^{+m} and C^{+m-n}, where m>n; and where a crystal or phase structure of the mineral includes one or more such species. In the net reaction presented in FIG. 11, F denotes reactive intermediates formed by catalyzed C—C bond scission in PAP, i.e, a reactive Fragment; and F—H denotes the product from quenching of F by H(equiv). The figure depicts a threefold point made previously, that (i) AR of F is limited, e.g., it does not consume the entirety of the fragment but ostensibly consumes only carbon associated the alkyl radical; (ii) the net yield of 5 H(equiv) depicted in FIG. 11 is available to quench other F; and (iii) AR of F to generate H(equiv) is self-regulating, e.g., it occurs only when F exist in the reaction mixture due to quantitative consumption of H(equiv) by quenching of F.

Reaction Temperatures and Energetics. PAP conversion in embodiments of the present invention occurs through the concurrent operation of three desirable reactions in a reaction mixture containing PAP in feedstocks and two agents added in appropriate quantities: a First Process Agent PA1, which is a catalyst; and water. The first desirable reaction is PA1-catalyzed C—C bond scission in polymer backbones, producing reactive intermediates, denoted as fragments F (Equation 7.1); the second is the combining of F with water to generate hydrogen equivalents, H(equiv) (Equation 7.2); and the third is quenching of other F by H(equiv) to obtain stabilized fragments F-H (FIG. 7.3). Neither of the first two reactions advances apart from the application of heat for reasons well understood by artisans: bond scission is endothermic (AH is positive); and likewise, aqueous phase reforming (APR) of all compounds is known to be endothermic. Although energetics of quenching of F by H(equiv) to form F—H are favored (AH has a large negative value), that is insufficient to drive the overall, desired conversion of PAP to stabilized, lower-MW product compounds.

Embodiments of the present invention overcome these inhibitions by applying temperatures at/above 390° C. where the catalyzed bond scission and AR of F proceed. Skilled artisans customarily explain this as a consequence of the entropic term in the Gibbs Free Energy equation (ΔG=ΔH−TΔS) overtaking the enthalpy term. Consistent with that is the observed correlation of conversion rates with temperature, because ΔG becomes progressively more negative with increasing temperature. Operational results evidence that quenching occurs readily at those temperatures, suggesting that at/above 390° C., both AR of F that generates H(equiv) and quenching of other F by the same are not rate-limiting.

Progressive, Conversion of PAP and PAP Fragments. Implied in discussion hereinabove is that particular embodiments effect conversion of PAP in feedstocks through scission that initially generates two reactive fragments F from a parent PAP, which, though smaller than the parent PAP, still are very large compared with the desired lower-MW product compounds. Those fragments then undergo further scission-quenching that generates ever-smaller F-H and continues iteratively until their average size in the reaction mixture corresponds to that required to meet quality criteria for the PAP conversion product, nonlimiting examples of which include average MW and/or average carbon number.

Initial Conversion. The depiction of PAP conversion kinetics in FIG. 4 shows that at low relative residence times, e.g, when t(rel) is about 0.05, hydrocarbons in the initial reaction mixture include: predominantly unconverted PAP, e.g., about 85 mole-percent; and about 10 to 15 mole-percent of lower-MW compounds B, C, and D. The significance of this Initial Conversion is that the reduction in molecular weight of PAP in the feedstock by one-fourth to one-fiftieth yields an Initial Conversion product (F-H)_{B} that is no longer a polymer melt but a liquid with low viscosity at process temperature. In particular embodiments Initial Conversion occurs at temperatures T(init) of 390 to 440° C. applied for residence times t(init) of about 0.1 to 1 hours. For reasons already discussed, the rate for Initial Conversion of PAP to (F-H)_{B} has a disproportionately greater impact on overall rates for conversion of PAP to the lower-MW products, (F-H)_{D}, because it is substantially lower than the rates for production of (F-H)_{C}, (F-H)_{D}. Particular embodiments will be described, which mitigate that impact.

In the chemical method, particular embodiments optionally add water to the initial reaction mixture while other particular embodiments do not. Even when not added, it is nevertheless present at low levels, e.g., in percentages from tenths to low-single-digit, being entrained in feedstocks or bound to minerals of PA1. In such cases, water in the initial reaction mixture is consumed in any/all of four modes: hydrolysis of any condensation polymers that contaminate the feedstock, e.g., polyesters and polyamides; AR of EG generated in hydrolysis of PET; AR of unquenched fragments F generated in the scission of C—C in polymer backbones; and AR of incipient coke. Embodiments that neglect to include water in the initial reaction mixture may seem to violate a core tenet of the present invention, e.g., the substantially quantitative quenching of F with autogenous H(equiv) to suppress formation of unsaturations in products from PAP conversion. Indeed, suppose by way of nonlimiting illustration that the feedstock contains 96% PE and 2% each of nylon and PET; the average carbon number of F-H generated in Initial Conversion is one-fiftieth that of PE in the feedstock; and the average carbon number of the PE is 50,000. The stoichiometric demand for water consumed in the three modes is, respectively, about 0.75%, 0.68%, and 0.10% w/w relative to feedstock. Thus, 1.5% water in feedstock is sufficient to meet the total demand in the three modes. However, if the amount is 1.4% or less, then because hydrolysis occurs readily at temperatures below about 300° C., all water will be consumed rapidly upon heating of the feedstock, well before reaching process temperatures at/above about 390° C. applied in particular embodiments. Consider finally that for the illustration, quantitative quenching in Initial Conversion requires only 0.14 H(equiv)/kg PE, corresponding to the stoichiometric demand given above as 0.1% w/w relative to feedstock. Absent the required H(equiv), and supposing that each pair of unquenched F yields one unsaturation, the corresponding bromine number (BN) is about 3.2 g bromine/100 sample, or about an order of magnitude lower than the total BN of the lower-MW products obtained by PAP conversion in particular embodiments. (That BN is a worst case that does not take into account H(equiv) generated from AR of EG when PET contaminates the feedstock.) If that contribution to total BN is consequential in view of offtake requirements for certain product applications, then embodiments may be configured and operated to maximize quenching of F in initial reaction mixtures.

By the same reasoning, particular embodiments will be described, which effect Initial Conversion at maximum T(init) of up to 440° C., even as the maximum temperature for Main Conversion is 430° C. That higher temperature in the former case elevates the rates for undesirable reactions to significant levels, where outcomes from such reactions are like those that attend thermolytic mechanisms and are a problematic feature of pyrolysis methods. If it also were applied in Main Conversion, the lower-MW product would exhibit inferior qualities characteristic of pyrolysis. But the effect is limited when a temperature of 440° C. is applied for residence times of short duration during Initial Conversion. Thus, in cases where the impact on final product quality of limited undesirable reactions during Initial Conversion is acceptable, embodiments that apply the higher temperature benefit from markedly higher throughput.

Main Conversion. The annotation Main Conversion in FIG. 4 indicates that lower-MW products are obtained through iterative scission-quenching of F-H generated in Initial Conversion at temperatures T(main) applied for residence times/(main). Because the quantity of scission events occurring in Main Conversion is about two to three orders of magnitude greater than that occurring in Initial Conversion, addition of water is not optional; it must be added to and dispersed in the main reaction mixture to obtain autogenous H(equiv) in quantities sufficient to quantitatively quench fragments F produced during Main Conversion.

Extent of Conversion. The descriptions of Initial and Main Conversion invoke numbers relating to the reduction in carbon numbers that attends PAP conversion to lower-MW products, e.g., those of F-H from Initial Conversion are about one-fourth to one-fiftieth of those for the parent PAP, and Main Conversion reduces the carbon number of those F-H by about two to three orders of magnitude to obtain lower-MW products, referred to as D or (F-H)_{D} in the detailed description of the kinetic framework. Given the large carbon number range spanned in PAP conversion, this progressive reduction of carbon number may be more conveniently expressed logarithmically as Extent of Conversion, as depicted in FIG. 12 by way of nonlimiting illustration wherein A is a high-density PE. The carbon number value given for A is representative but will vary widely for PE in feedstocks due to the diversity of sources represented in waste plastic feedstocks; and the carbon number distribution of A will be even wider. The carbon number range for B relates to that for A by factors of 4 and 50, corresponding to the definition of Initial Conversion; the values given for D relate approximately to the range that is desirable for the lower-MW conversion product; and values for C are mathematically determined by those defined for B and D.

Carbon numbers for A will vary according to polymer grade, e.g., LLDPE, LDPE, MDPE, HDPE, and UHMWPE; and polymer type, e.g., PP, PS, and PIB, each of which also has a plurality of grades. An additional factor is use history of the waste plastic, as PAP may degrade during the production and use of articles, and during post-use collection and handling that precedes conversion in embodiments hereof. In all cases, the Extent of Conversion during Initial Conversion is about 0.6 to 1.7 and average carbon numbers for D are about 10 to 50, as given in Table 12. However, by way again of nonlimiting illustration, if the average carbon number of A were lower, e.g., in the range of about 2,000 to 10,000, then the ranges for the cumulative Extent of Conversion for B and C will be smaller and the overall Extent of Conversion for D will be about 2.3 to 3.0. Yet the overall kinetic framework would remain valid and contours for plots of their concentrations over time would be substantially the same as that depicted in FIG. 4.

### Process Method

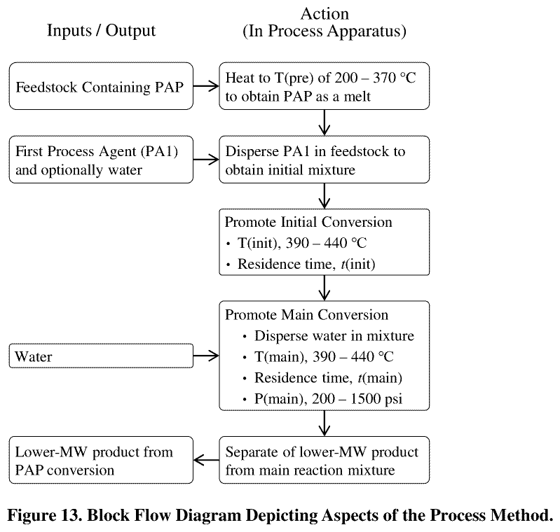

In accordance with the foregoing detailed description of the chemical method, and referring now to FIG. 13, the process method in embodiments of the instant invention includes apparatus configured to effect conversion of polyaddition polymers (PAP) into lower-MW compounds in steps that combine and heat feedstocks with PA1 and water, as appropriate. Thus, particular embodiments include apparatus configured to,
(a) separately receive the PAP-containing feedstock, a First Process Agent PA1, and water;
(b) receive and heat the feedstock to a preliminary temperature T (pre) of about 200 and 370° C., to lower its viscosity and obtain a liquid or melt, as the case may be;
(c) combine PA1 and optionally water with the feedstock liquid or melt, and promote the contacting and continuous dispersion of PA1 and water, as the case may be, into the feedstock to obtain an initial mixture;
(d) heat the initial mixture to a temperature T(init) of about 390° C. to 440° C. and continuously disperse PA1 therein for about 0.1 to 1 hours to promote scission of carbon-carbon bonds in the PAP backbone or chain, by operation of PA1, thereby obtaining an initial reaction mixture containing polymer fragments F and/or F-H, as the case may be, and achieving Initial Conversion wherein the Extent of Conversion is about 0.6 to 1.7;
(e) combine water with the reaction mixture, apply continuous mixing to disperse it and components in the process mixture to obtain a main reaction mixture, and maintain the same at a temperature T(main) of about 390 to 430° C. and a pressure P (main) of about 200 to 1500 psi for a residence time t(main) of about 0.5 to 5 hours to promote Main Conversion;
(f) control (b)-(e) to maximize throughput and product yield in consideration of desired product quality;
(g) apply means suitable for removing desired product compounds from the product mixture, where condensed-phase separation and distillation are nonlimiting examples of such means.

In particular embodiments, (a)-(e) are performed nominally sequentially whereas other particular embodiments execute (d)-(e) nominally concurrently while yet other particular embodiments effect (b)-(e) nominally concurrently. In other particular embodiments, water is not added in (c) due to presence of quantities in feedstocks and/or PA1 that are sufficient to meet the stoichiometric demand in Initial Conversion for hydrolysis of polyesters and polyamides, and in AR of EG from PET, of F, and of incipient coke, which produce H(equiv) autogenously. And in yet other particular embodiments, water is not added in (c) even if amounts otherwise available are stoichiometrically limiting in respect of H(equiv) production, as the resulting contribution to unsaturation levels in the lower-MW product are substantially inconsequential.

As regards element (f) of the process method, quality criteria including average molecular weight or average carbon number and/or carbon number distribution serve as measures of the Extent of Conversion which inform operational adjustments to control severity of the conversion process, where severity relates quantitatively to residence time, temperature, catalyst selection (and catalyst activity), and catalyst concentration. An additional criterion beyond process severity concerns yield, e.g., the amount of desired lower-MW compounds obtained, relative to PAP in the feedstock.

Single-Pass versus Multiple-Pass Conversion. The chemical method description identifies PAP conversion as occurring through C—C bond scission, beginning with Initial Conversion in the parent PAP of feedstocks and continuing progressively in Main Conversion to produce ever-smaller reactive fragments F that are quenched with H(equiv) to obtain stabilized fragments F-H. Skilled artisans will recognize readily that implementing the process method in embodiments that employ a solitary batch reactor will generate a mixture of product compounds with a distribution of molecular weights (or carbon numbers) determined by process severity. In general, average MW diminishes with increasing severity; and increasing severity directed at driving product compounds toward ever-lower-MW results in lower liquid yields due to increasing production of gaseous product compounds, notwithstanding the function in embodiments hereof that limits overconversion of lower-MW product compounds. Accordingly, exemplary embodiments described hereinbelow maximize liquid yields while mitigating yield loss through overconversion, by optimizing configuration and operation of process apparatus that control Initial Conversion, Main Conversion, and product recovery.

Pressure. In particular embodiments, process pressures in the range of about 200 to 1500 psi are generated autogenously by water added in excess of that consumed in hydrolysis various and AR reactions described previously; by lower-MW compounds generated by PAP conversion, the vapor pressure of some being significant at process temperatures applied in Main Conversion; by CO₂ produced in AR of F, EG, and incipient coke; and by H₂S produced through hydrodesulfurization when PAP is TR. Moreover, while wishing to not be bound by any particular theory of operation, the corresponding partial pressures of water are thought to suppress both cyclization/aromatization that compromises product quality.

### DESCRIPTION OF EMBODIMENTS

General Description. The present invention consists of chemical methods realized through process methods, both configured in embodiments of the present invention to effect the conversion of polyaddition polymers (PAP) contained in lower-value feedstocks to obtain higher-value products containing compounds of lower molecular weight, wherein:
(a) the feedstocks include, (i) post-use plastics whose PAP include polystyrene (PS) and polyalkylenes, where the latter includes polyisobutylene (PIB) and polyolefins (PO), which are (PE) and polypropylene (PP), or (ii) tire rubber (TR) whose PAP includes the hydrocarbon fraction synthesized with natural rubber and synthetic polymers derived from petroleum including styrene, butadiene, isoprene, ethylene, and propylene;
(b) PAP conversion occurs in a reaction mixture by the scission of carbon-carbon bonds (C—C) in the PAP backbone, chain, matrix, or network that yields fragments F, which are reactive intermediates;
(c) the C—C scission occurs by agency of a First Process Agent (PA1), a catalyst based on naturally-occurring minerals including (i) aluminosilicate compounds and aluminum silicate compounds such as montmorillonite clay, bentonite clay, kaolinite clay, and laterite clay; and/or (ii) mordenite; and/or (iii) hematite and hematite clay; and/or (iv) magnetite;
(d) PA1 is selected in respect of availability, cost, and catalytic activity;
(e) water in the reaction mixture combines with F through in situ aqueous reforming (AR) to generate hydrogen equivalents, H(equiv), that quench other F to obtain stabilized PAP fragments F-H, where H(equiv) is one or more intermediate species able to impart hydrogen equivalents to F and where the production of H(equiv) is autogenous and self-limiting;
   and wherein the process apparatus is configured to,
(f) receive the feedstock and heat it to a preliminary temperature T (pre) of between about 200 and 370° C. to lower its viscosity to obtain a liquid or melt that is predisposed to combining with PA1 and optionally water;
(g) receive PA1, and optionally water, and obtain an initial mixture by dispersing the same into the feedstock and promoting the contacting of PAP therein with PA1;
(h) heat the initial mixture to a process temperature T(init) of about 390 to 440° C., and to continuously disperse components therein for a residence time t(init) of about 0.1 to 1 hours to promote scission in the PAP, to obtain an initial reaction mixture containing PAP fragments F and/or F-H, as the case may be, and thereby achieve Initial Conversion wherein the Extent of Conversion is about 0.6 to 1.7.
(i) combine water with the reaction mixture and continuously disperse it and mixture components;
(j) maintain the mixture at a temperature T(main) of about 390 to 430° C. under an applied pressure of P (main) of about 200 to 1500 psi for a residence time t(main) of about 0.5 to 5 hours to promote Main Conversion, where pressures accrue autogenously and where T(main) and t(main) are selected in respect of catalyst activity and concentration, and of desired yield and quality of lower-MW hydrocarbons, which quality relates to average carbon number, or Extent of Conversion;
(k) isolate from the reaction mixture the desired lower-MW compounds produced by PAP conversion, where the means of isolation include condensed-phase separation based on density or solubility and vapor-phase separation based on boiling point (distillation).

Particular embodiments are now described, which examine aspects of elements enumerated in the foregoing general description of embodiments.

Feedstocks: Polyaddition Polymers in Waste Plastics. Feedstocks in particular embodiments are in the form of pellets, powders, granules, flakes, pellets, chips, and the like. They contain, in particular embodiments, polyolefins at levels of about 75% to 98%, which polyolefin fraction contains 0% to 100% PE while the balance is PP. The composition of the non-polyolefin fraction is variable and consists of contaminants of two types: plastics made of polymers other than polyolefins; and non-polymeric materials. Generally, the amounts and types of both are due to the diversity of municipalities' plastic waste streams and the limitations of mechanical sorting technologies applied to produce waste plastic streams enriched in one or another polymer, e.g., PET, polyolefins, and PS, and minimizing in them the amounts of others such as nylons, polyurethanes, and PVC. Typically, those enriched steams also contain low-percentage levels of other contaminants including paper and compounds in plastics such as colorants and fillers, plasticizers, and agents added to facilitate plastic extrusion. In other particular embodiments, the waste plastic feedstock consists of PS at levels of about 75% to 98%, with the balance consisting of polymeric contaminants such as PE, PP, PETE, nylons, polyurethanes, and PVC.

Feedstocks: Tire Rubber. In other particular embodiments the feedstock consists of granules of rubber from end-of-life transportation tires. Tire rubber is synthesized from both natural rubber and diverse prepolymers derived from petroleum including styrene, butadiene, isoprene, ethylene, and propylene. As such, TR is a polyaddition polymer but unlike embodiments where PAP in feedstocks are substantially homopolymeric, e.g., PE, PP, PIB, and PS, TR may be viewed as heteropolymeric, including a plurality of different PAP such that conversion of TR by particular embodiments yields products that are complex mixtures of hydrocarbons whose composition relates to the diversity of compounds from which tire rubber is synthesized and includes paraffinic, isoparaffinic, and aromatic functionalities.

In work by the inventors to configure embodiments of the present invention for conversion of TR, they observed (i) a diminished role of PA1 for achieving the required process severity, compared with other PAP such as PS and polyalkylenes; and (ii) a benefit in increasing the total amount of added water to about 20% w/w relative to feedstock. The latter is attributable to the absorption of water by carbon black, which diminishes its availability for other purposes enumerated hereinbelow. The lower requirement for PA1 is apparently due to the catalytic activities of one or more inorganic additives in TR formulations, which include carbon black, zinc oxide, titanium dioxide, and clay in total amounts typically around 30%. Accordingly, the process severity applied in particular embodiments includes selection of the quantities of added PA1, T(main), and t(main) in respect of conversion rates exhibited.

PA1 (Catalyst). In particular embodiments, PA1 consists of materials based on naturally-occurring minerals including, by way of nonlimiting example, one or more taken from the group consisting of or derived from: naturally-occurring aluminosilicate compounds and aluminum silicate compounds including montmorillonite clay, bentonite clay, kaolinite clay, hematite clay, mordenite, hematite clay, and laterite clay; hematite and hematite clay; and magnetite. As naturally-occurring materials, their structures, e.g., crystal frameworks and/or mineral phases, are diverse, as are their chemical compositions, where an aspect of the latter includes variability in hydration of the structures. Such diversity affects their activity in promoting C—C scission in PAP, which in turn requires that the amount of added PA1 must be taken into account when configuring particular embodiments in respect of other process parameters that determine severity including the susceptibility of a given PAP to undergo C—C scission. Activity variations notwithstanding, the amount of added PA1 in reaction mixtures of particular embodiments is about 1% to 4% w/w relative to the feedstock.

All of the aforementioned PA1 are derived directly from naturally occurring minerals, yet they all are prepared minerals, e.g., they are conditioned industrially through one or more operations, nonlimiting examples of which include, (i) cleaning and grinding of the raw material; (ii) further mechanical processing that obtains the mineral in the form of granules or powders with some particular particle size range; (iii) mild heating to remove unbound water or calcination to remove carbonates; (iv) washing with water to remove water-soluble salts or acid leaching to remove carbonate minerals; (v) activation by treatment with acids; and (vi) modifying chemical and/or porosity characteristics with one or more metals and/or other elements applied through an impregnation process, e.g., deposition or insertion, such as pillaring. In particular embodiments, the metals applied include one or more transition metals from groups 3-12 and from periods 4 and 5 of the periodic table of the elements, including by way of nonlimiting example V, Ti, Fe, Mn, Cu, Y, Zr, and Mo; and other elements applied include (a) one or more post-transition metals from Groups 12 and 13, including Al, Ga, In, and Sn, and (b) one or more lanthanides including La and Ce, and (c) Si.

Higher-Value Products from Lower-Value Feedstocks. In particular embodiments when PAP in feedstocks is PS or polyalkylenes, nonlimiting examples of higher-value products include hydrocarbons having carbon numbers in the range of about 6 to 35, which are substantially saturated apart from aromatic functionality present in feedstock PAP, and whose structures relate substantially to those in the parent PAP from which they were derived. And for particular embodiments where the feedstock is TR, the higher-value products include by way of nonlimiting example hydrocarbons having carbon numbers in the range of about 6 to 35, which are substantially saturated apart from aromatic functionality present in feedstock PAP, and which have diverse structures related to the aromatic and aliphatic structures present in the hydrocarbon fraction of TR. In particular embodiments where PAP in feedstocks includes PE, PP, PS, and PIB, quality parameters of principal importance are average carbon number, carbon number distribution, and bromine number (BN), the latter being a standard measure of unsaturation expressed as grams bromine (Br₂) per 100 g sample. In cases covered below under Process Apparatus, PAP conversion products consist of compounds whose carbon numbers range from about 6 to 35 while average carbon number and the lower- or higher end of the distribution within that range may be tuned by adjusting process severity. Such tuning beneficially permits carbon number distribution to be skewed in respect of commercial end-use.

Product compounds from PE conversion consist substantially of saturated straight-chain alkanes while products from PP are substantially methyloligopropylene compounds (MOP), which are alkanes that bear a methyl group on every other carbon of the molecular backbone, as in the parent PAP. In other particular embodiments, the PAP is a poly(isobutylene), or PIB, a dimethyl analog of PP in which every second carbon in the polymer backbone bears two methyl groups and the lower-MW conversion products may be described as containing methyloligoisobutylene compounds (MOB). And products obtained from PS conversion include mono-substituted benzene compounds such as toluene and ethyl benzene; and di- and tri-phenyl alkanes, for which precursor F are depicted in FIG. 7.

In all cases, the products are substantially saturated and contain single-digit percentages of alkene functionality and of aromatic functionality, excluding phenyl groups in styrenic moieties of PAP. Where PAP includes polyolefins, the products' higher value relates to their suitability as feedstocks to naphtha crackers as a spoke in the circular economy. When PE is the PAP in feedstocks, the product hydrocarbons' higher value relates to their usefulness in commercial and industrial applications requiring high-purity paraffinic basestocks. For MOP and MOB compounds produced in embodiments where the PAP in feedstocks is PP or PIB, respectively, their higher value relates generally to that of isoparaffinic compounds that are useful in a wide range of applications including industrial cleaning and degreasing solvents, paints and coatings, adhesives, lubricant basestocks, and personal care products. The value of products from embodiments where the PAP in feedstocks is PS relates to similar uses while circularity via ethylbenzene as a feedstock for production of styrene is particularly compelling. When the feedstock is TR, the product hydrocarbon mixture is correspondingly complex and includes paraffinic, isoparaffinic, and aromatic functionalities; their higher value relates principally to their solvent properties and the possibility for dilution in feedstocks to naphtha crackers.

Process Apparatus: Single-Pass Conversion. In particular embodiments configured to apply the process method to realize the chemical method, the process apparatus consists of a single continuously-stirred tank reactor (CSTR) configured to (i) receive the feedstock; (ii) heat the feedstock to between about 200 and 370° C., to lower the viscosity of the feedstock and obtain a liquid or melt; (iii) receive PA1 selected in respect of cost, availability, and catalytic activity; (iv) promote dispersal of PA1 into and throughout the feedstock and promote its contacting with PAP therein to obtain an initial mixture; (v) receive water in an amount appropriate to support hydrolysis of polyester and polyamide contaminants in the feedstock, aqueous reforming (AR) of ethylene glycol from PET hydrolysis, AR that generates H(equiv) from fragments F produced in C—C scission, and AR of incipient coke in the reaction mixture; (vi) disperse the added water into and throughout the initial mixture by stirring to obtain an initial reaction mixture; (vii) heat the mixture to a process temperature between 39° and 430° C. to promote Initial Conversion of PAP by catalyzed C—C scission that generates reactive intermediate fragments F and also promotes AR of F to generate H(equiv) that quench other F and obtain stabilized fragments F-H; (viii) maintain pressure over the reaction mixture at between about 200 and 1500 psi; and (ix) maintain the reaction mixture at the process temperature and pressure for a residence time of about 1 to 8 hours, to promote Main Conversion. A CSTR suitable for particular embodiments described is a Parr 4570 HP/HT and pressure is maintained at selected levels through the operation of a conventional pressure regulator installed on the CSTR.

The residence time is selected in respect of other parameters related to process severity, which bears on the simultaneous objectives of maximizing yields of lower-MW compounds; minimizing yield loss due to overconversion that generates gaseous byproducts at the expense of liquid yields; and maximizing product quality parameters that determine product value. Subsequently, in particular embodiments the desired lower-MW compounds in the product mixture are isolated by density-based separation and/or by liquid-liquid extraction that removes catalyst, water, polar reaction byproducts, and non-hydrocarbon contaminants from the feedstock. Other particular embodiments recover lower-MW hydrocarbon product compounds by distillation, where those with carbon numbers below about 25 distill at/below about 350° C. under ambient pressure while those with carbon numbers below about 35 may be recovered by distillation at a reduced pressure of about 35 mm Hg. Particular embodiments recover lower-MW products by distillation directly from the reaction mixture after cooling the same to 350° C. and by configuring the reactor with an overhead port that may be opened to permit flow of product vapor to a column with a condenser suitably configured to recover distilled compounds as a liquid.

When the PAP in particular embodiments is PS and/or polyalkylenes and the process apparatus includes a CSTR configured and operated as described to effect single-pass conversion, distillation recovers the liquid product mixture containing lower-MW hydrocarbons in amounts of about 60% and 80% relative to PAP in the feedstock, which have carbon numbers in the range of about six to 35 while the balance consists of stabilized fragments whose carbon numbers are greater than about 35.

Increased Yields through Multi-Pass Conversion. Net yields of lower-MW hydrocarbons in other particular embodiments increase the net yields to about 90% relative to PAP in the feedstock, through the further conversion of non-distillable PAP fragments with carbon numbers greater than about 35, which remain in the distillation residue together with the PA1. In particular embodiments, that residue is recycled back into the CSTR. And in yet other particular embodiments, the residue remaining after distillation flows into a second reactor where conversion continues with addition of water. Skilled artisans will readily identify additional possibilities to configure process apparatus in ways to maximize both yields and process throughput, including semi-batch; plural CSTRs in series; tubular reactors; and a product recovery section including distillation column in series with the one or more reactors.

Increased Efficiency with Multiple Subsystems. In embodiments that effect single- and multiple-pass conversion in CSTRs, the conversion kinetics largely align with those portrayed in FIG. 4 of the kinetic framework: Initial Conversion dominates early at low relative residence times while Main Conversion quickly ensues, wherein molar concentrations of lower-MW products D overtake those of unconverted PAP, represented as A. For reasons detailed previously, conversion rates of A diminish after Initial Conversion, which constrains throughput in commercial implementations of the chemical method. But the kinetic framework also suggests a way to overcome that constraint: substantially effect Initial and Main Conversion separately, in separate reactor subsystems.

Accordingly, particular embodiments consist of a system configured promote Initial and Main Conversion in distinct subsystems wherein, (i) a first subsystem (Subsystem1) receives the feedstock and heats it to about 200 to 370° C., to obtain PAP therein as a melt; (ii) Subsystem1 further combines the melt with PA1, and optionally with water, and obtains an initial mixture by dispersing the same into the feedstock melt and promoting contacting therebetween; (iii) Subsystem1 further heats the initial mixture to a process temperature of about 390 to 440° C. and continuously disperses components therein for about 0.1 to 1 hours to promote scission of carbon-carbon bonds in the PAP, by operation of PA1, thereby obtaining an initial reaction mixture containing polymer fragments F and/or F-H, as the case may be, and achieving Initial Conversion wherein the Extent of Conversion is about 0.6 to 1.7; (iv) a second subsystem (Subsystem2) is communicably coupled to the first and configured to receive flow therefrom of the product mixture from Initial Conversion, and to combine the same with water sufficient to meet stoichiometric demand and autogenously achieve the required pressure over the reaction mixture and to obtain a main reaction mixture and promote Main Conversion, as described previously; and (v) a third subsystem (Subsystem3) is the communicably coupled to the second and configured to receive flow therefrom of the product mixture from Main Conversion and to recover the desired lower-MW compounds contained in the same through distillation. A CSTR suitable for such embodiments is a Parr 4570 HP/HT and pressure is maintained at selected levels through the operation of a conventional pressure regulator installed on the CSTR.

In particular embodiments, Subsystem 1 has the form of a first tank configured to receive and heat the feedstock to 200 to 370° C., receive PA1 and optionally water, and disperse those in the feedstock; and a second tank configured to further heat the initial mixture to about 390 to 440° C. to promote Initial Conversion. Delivery of feedstock into the first tank is direct and substantially continuous by means of devices known to skilled artisans for rapid, sequential dosing of pellets, powders, granules, and the like, nonlimiting examples of which include, solid dosing valves, rotary dosing valves, bulk solids valves, volumetric dosing valves, gravimetric dosing valves, and pneumatic conveying systems sometimes referred to as powder transfer systems; or an extruder such as a Leistritz ZSE 27 MAXX.

In other particular embodiments, Subsystem 1 is an extruder or plural extruders configured in series to effect features (f)-(h) of the General Description, where the configuring relates to variables including but not limited to screw element design, the number of screws, screw speed, length-to-diameter ratio, relative direction of screw rotation (when more than one), the number and length of zones in the extruder, the temperature within each such zone, and residence time in each zone. Where Subsystem 1 is two extruders in series, the role of the first is to generate the polymer melt by heating the feedstock to about 200 to 370° C. and optionally combine and disperse PA1 thereinto; and that of the second is to combine the extrudate received from the first with PA1, if not added previously, and to promote Initial Conversion through the contacting between PA1 and PAP and by heating the initial mixture to a temperature of about 390 to 440° C. Embodiments that employ extrusion in Subsystem1 benefit from the high mass- and heat-transfer for which particular configurations of extruders are well known, e.g., promoting Initial Conversion at high rates due to high efficiency in the heating of feedstocks, their mixing with PA1, and the contacting between PA1 and PAP in feedstocks.

In particular embodiments, Subsystem2 is a CSTR configured to enable Main Conversion as described previously at applied temperatures of about 390 to 430° C., where the flow rates of the input to Subsystem1 and output from Subsystem3 are selected to (i) maintain the main reaction mixture at a volume of about 25% and 95% of the volume of the CSTR; and (ii) provide a mean residence time for the main reaction mixture of between about 0.5 and 5 hours, to effect Main Conversion. Compared with embodiments configured with a single CSTR, in which Initial and Main Conversion occur in the selfsame reaction mixture, embodiments with subsystems configured to promote Initial and Main Conversion substantially separately were found by the inventors to reduce net residence time by more than about 20%, corresponding to an increase in throughput by greater than about 25%.

Subsystem3 in particular embodiments receives from Subsystem2 the product from Main Conversion flowing at a rate suitable to maintain the reaction mixture therein at the desired level for the desired residence time. In Subsystem3, the lower-MW product compounds are recovered by distillation at temperatures and pressures suitable to obtain products with the desired carbon number range, as already described. And as described in connection with multi-pass conversion, yields of the recovered product may be increased by further conversion of heavier hydrocarbons retained in stillbottoms, e.g., by conversion in a subsequent CSTR or by recycling of the residue back into the CSTR.

Facile Conversion of Crosslinked PAP. Embodiments that include a Subsystem 1 comprising one or more extruders are highly advantageous when PAP in the feedstock includes crosslinked polymers such as PEX and TR. Although such polymers do not melt, particular embodiments enable their efficient Initial Conversion through co-extrusion with other, non-crosslinked PAP. In such embodiments, Subsystem 1 effects Initial Conversion by (i) combining PEX or TR granules with (a) another type of PAP, e.g., HDPE, in a ratio of about 1.5:1 to 1:1.5 w/w, and with (b) PA1, as appropriate; and (ii) heating that initial mixture to about 390 to 440° C. to promote Initial Conversion. Through the operation of an extruder in such embodiments, the co-extruded PAP melts and serves as a medium wherein the PEX granules and PA1 disperse to obtain an initial mixture; then Initial Conversion occurs through the further operation of the extruder to efficiently promote heating to about 390 to 440° C. and mass transfer that achieves the contacting of PA1 with PEX or TR and the other PAP. Other particular embodiments directed toward conversion of TR apply PS as the non-crosslinked PAP co-extruded with TR, due to its higher solvency strength and chemical commonality with SBR (styrene-butadiene-rubber), which TR formulations typically contain at levels of about 50%.

The possibility exists to configure particular embodiments that co-convert feedstock mixtures that include bulk quantities of two or more PAP taken from the group consisting of PE, PP, PS, and PIB; or alternatively TR and/or PEX and one or more of PE, PP, PS, and PIB. Yet, the lower-MW product derived from such mixtures will be complex as regards the amounts and types of hydrocarbons. Although their dilution in naphtha may permit their conversion by cracking to ethylene and propylene, the diversity of chemical functionality would otherwise limit their use apart from solvents and fuels.

Water: Its Roles and Required Quantities. Water serves a fourfold role in the chemical and process methods realized in particular embodiments. In two cases, water is a reactant: in hydrolysis of feedstock contaminants, e.g., PETE and nylons; and in AR of F, EG from PET, and incipient coke, all of which generate H(equiv). In the other two roles, water is a chemo-physical agent. Due its high vapor pressure at temperatures applied to the main reaction mixture, the addition thereto of quantities in excess of those consumed in reactions is the principal means for achieving and controlling pressures required in Main Conversion. Second, the high heat capacity and coefficient of thermal conductivity of water, and its high mobility at elevated temperatures applied in particular embodiments, make it an effective agent of heat transfer into and through the reaction mixture and of mass transfer of components in the same. Accordingly, particular embodiments add quantities of water beyond what accompanies feedstocks and PA1 such that the total amount in CSTRs employed in Main Conversion is about 10% and 15% relative to the feedstock, where addition in particular embodiments is at a rate corresponding to the rate of feedstock flow through the process. And where the feedstock is TR, particular embodiments add water in amounts of about one and five times that of the feedstock.

### EXAMPLES

### Example 1. Waste PE in a Closed CSTR

The process apparatus consisted of a Parr 4575 HT/HP stirred, closed tank reactor with a volume of 1 L. The feedstock charged into it was waste plastic that contained about 95% PE and had a melt index 12 g/10 min (192° C./2.16 kg). The catalyst was bentonite clay, which together with water was charged into the reactor in amounts of 2.0% and 5.0% w/w, respectively, relative to the feedstock and the total mixture occupied 50% of the reactor volume. The reactor heated the mixture to 325° C. to obtain PE in the form of a low-viscosity melt and, by operation of the stirrer at 200 rpm, dispersed catalyst and water therethrough to obtain an initial mixture and promote contacting of PE with the catalyst. Subsequently, under continued stirring, the initial mixture was heated to a process temperature of 420° C. to obtain an initial reaction mixture and maintained there for a residence time of 2 hours to promote (i) heat- and mass transfer within the reaction mixture; and (ii) Initial Conversion and Main Conversion of PE through (a) catalytic C—C scission to generate reactive fragments F, (b) autogenous in situ production of H(equiv) by limited AR of F, (c) quenching of other F by H(equiv) to obtain stabilized fragments F-H; and (d) iterative scission-quenching of such fragments to produce progressively-smaller F-H. At the process temperature, pressures in the reactor rose autogenously to a maximum between 1200 and 1400 psi due to (i) the vapor pressure of lower-MW product compounds and of water added in excess of that required to meet the stoichiometric demand in (a) hydrolysis of contaminant PET and nylon, if present, and (b) AR of F, ethylene glycol from PET (if present), and incipient coke; and (ii) carbon dioxide generated by AR. After cooling and relief of residual pressure, the hydrocarbon phase was isolated from the catalyst and water-soluble byproducts in the product mixture by liquid-liquid extraction into hexane. Removal of hexane recovered the product mixture in 51% yield, relative to the feedstock, which consisted substantially of saturated straight-chain alkanes whose aggregated bromine number (BN) was 17±1 g bromine/100 g sample and whose distribution in carbon number subfractions was: ≤20, 26%; 21 to 30, 26%; 31 to 40, 15%; >40, 33%.

### Example 2. Waste PE in a Closed CSTR

The process apparatus consisted of a Parr 4575 HT/HP closed, stirred tank reactor with a volume of 0.5 L and the feedstock charged into it was the same as that used in Example 1. The catalyst was aluminum pillared montmorillonite clay, which together with water was charged into the reactor in amounts of 2.0% and 7.5% w/w, respectively, relative to the feedstock and the total mixture occupied 25% of the reactor volume. The reactor heated the mixture to 325° C. to obtain PE in the form of a low-viscosity melt and, by operation of the stirrer at 200 rpm, dispersed catalyst and water therethrough to obtain an initial mixture and promote contacting of PE with the catalyst. Subsequently, under continued stirring, the initial mixture was heated to a process temperature of 420° C. to obtain an initial reaction mixture and maintained there for a residence time of 2 hours to promote (i) heat- and mass transfer and (ii) Initial Conversion and Main Conversion of PE by reactions and mechanisms described for Example 1. At the process temperature, pressures in the reactor rose autogenously to a maximum between 1000 and 1200 psi due to the factors described in Example 1. After cooling and relief of residual pressure, the light hydrocarbon fraction was isolated from the water, catalyst, and wax fractions as the supernatant following centrifugation of the product mixture; and the wax fraction was separated from the catalyst and water-soluble byproducts in the aqueous fraction by liquid-liquid extraction into hexane. The lower-MW hydrocarbon product corresponding to the combined liquid hydrocarbon and wax fractions was obtained in a yield of 82%, relative to the feedstock, and consisted substantially of saturated straight-chain alkanes whose aggregated BN was 27±0.2 g bromine/100 g sample and whose distribution in carbon number subfractions was: ≤20, 70%; 21 to 30, 13%; 31 to 40, 6%; and >40, 11%.

### Example 3. Waste PE in a Closed CSTR

The process apparatus consisted of the same reactor used in Example 2. The feedstock charged into it was waste plastic that contained about 95% PE and had a melt index 12 g/10 min (192° C./2.16 kg). The catalyst was mordenite, which together with water was charged into the reactor in amounts of 2.0% and 12.5% w/w, respectively, relative to the feedstock and the total mixture occupied 12.5% of the reactor volume. The reactor heated the mixture to 325° C. to obtain PE in the form of a low-viscosity melt and, by operation of the stirrer at 200 rpm, dispersed catalyst and water therethrough to obtain an initial mixture and promote contacting of PE with the catalyst. Subsequently, under continued stirring, the initial mixture was heated to a process temperature of 420° C. to obtain an initial reaction mixture and maintained there for a residence time of 2 hours to promote (i) heat- and mass transfer and (ii) Initial Conversion and Main Conversion of PE by reactions and mechanisms described for Example 1. At the process temperature, pressures in the reactor rose autogenously to a maximum between 800 and 1000 psi due to the factors described in Example 1. After cooling and relief of residual pressure, the light hydrocarbon fraction was isolated from the water, catalyst, and wax fractions as the supernatant following centrifugation of the product mixture; and the wax fraction was separated from the catalyst and water-soluble byproducts in the aqueous fraction by liquid-liquid extraction into hexane. The lower-MW hydrocarbon product corresponding to the combined liquid hydrocarbon and wax fractions was obtained in a yield of 78%, relative to the feedstock, and consisted substantially of saturated straight-chain alkanes whose aggregated BN was 41±1 g bromine/100 g sample and whose distribution in carbon number subfractions was: ≤20, 73%; 21 to 30, 11%; 31 to 40, 8%; and >40, 8%.

### Example 4. Waste PP in a Closed CSTR

The process apparatus consisted of the same reactor used in Example 2. The feedstock charged into it was waste plastic that contained about 95% PP and had a melt index 13 g/10 min (230° C./2.16 kg). The catalyst was montmorillonite clay, which together with water was charged into the reactor in amounts of 2.0% and 10% w/w, respectively, relative to the feedstock and the total mixture occupied 25% of the reactor volume. The reactor heated the mixture to 325° C. to obtain PP in the form of a low-viscosity melt and, by operation of the stirrer at 200 rpm, dispersed catalyst and water therethrough to obtain an initial mixture and promote contacting of PP with the catalyst. Subsequently, under continued stirring, the initial mixture was heated to a process temperature of 395° C. to obtain an initial reaction mixture and maintained there for a residence time of 2 hours to promote (i) heat- and mass transfer and (ii) Initial Conversion and Main Conversion of PP by reactions and mechanisms described for PE in Example 1. At the process temperature, pressures in the reactor rose autogenously to a maximum between 800 and 1000 psi due to the factors described in Example 1. After cooling and relief of residual pressure, the light hydrocarbon fraction was isolated from the water, catalyst, and wax fractions as the supernatant following centrifugation of the product mixture; and the wax fraction was separated from the catalyst and water-soluble byproducts in the aqueous fraction by liquid-liquid extraction into hexane. The lower-MW hydrocarbon product corresponding to the combined liquid hydrocarbon and wax fractions was obtained in a yield of 82%, relative to the feedstock, and consisted substantially of methyloligopropylene alkanes whose aggregated BN was 56±1 g bromine/100 g sample and whose distribution in carbon number subfractions was: ≤20, 72%; 21 to 30, 12%; 31 to 40, 6%; and >40, 9%.

### Example 5. Waste Tire Rubber in a Closed CSTR

The process apparatus consisted of the same reactor used in Example 1. The feedstock charged into it was rubber granules sourced from end-of-life tires, which consisted of approximately 70% polymeric hydrocarbons (rubber), where the balance was inorganic fillers. The feedstock was charged into the reactor together with 20% water w/w relative to the feedstock and the total mixture occupied about 15% of the reactor volume. The reactor heated the mixture to 325° C. and, by operation of the stirrer at 200 rpm, dispersed the granules and water to obtain an initial mixture. Subsequently, under continued stirring, the initial mixture was heated to a process temperature of 410° C. to obtain an initial reaction mixture and maintained there for a residence time of 2 hours to promote (i) heat- and mass transfer and (ii) Initial Conversion and Main Conversion of the rubber by reactions and mechanisms described for PE in Example 1. At the process temperature, pressures in the reactor rose autogenously to a maximum between 1000 and 1200 psi due to (i) the vapor pressure of lower-MW product compounds, and of water added in excess of that required to meet the stoichiometric demand in AR of F, of EG from any contaminant PET, and of incipient coke; (ii) carbon dioxide generated by AR; and (iii) H₂S formed in desulfurization reactions. After cooling and relief of residual pressure, the hydrocarbon fraction was isolated by phase separation and extraction in hexane from the product mixture containing inorganic fillers and the aqueous phase containing water-soluble byproducts from the rubber. The lower-MW hydrocarbon product was obtained in a yield of 55%, relative to the polymer component of the feedstock, and consisted of diverse hydrocarbon compounds with alkyl and aromatic functionality, but which were otherwise substantially saturated, and whose aggregated BN was 32±1 g bromine/100 g sample and whose distribution in carbon number subfractions was: ≤20, 61%; 21 to 30, 20%; 31 to 40, 7%; and >40, 12%.

### Example 6. Mixed Polyolefins in Waste Plastic, in a Closed CSTR

The process apparatus consisted of the same reactor used in Example 2. The feedstock charged into it was waste plastic containing PE and PP in a total amount of about 85%, where the balance included about 6% PET, 1% PS, 1% nylon, 3% paper, and 4% inorganics. The catalyst was bentonite clay, which together with water was charged into the reactor in amounts of 2.0% and 12.5% w/w, respectively, relative to the feedstock and the total mixture occupied 12.5% of the reactor volume. The reactor heated the mixture to 325° C. to obtain the polymers in the feedstock in the form of a low-viscosity melt and, by operation of the stirrer at 200 rpm, dispersed catalyst and water therethrough to obtain an initial mixture and promote contacting of PE and PP with the catalyst. Subsequently, under continued stirring, the initial mixture was heated to a process temperature of 410° C. to obtain an initial reaction mixture and maintained there for a residence time of 2 hours to promote (i) heat- and mass transfer and (ii) Initial Conversion and Main Conversion of PE and PP by reactions and mechanisms described for PE in Example 1. At the process temperature, pressures in the reactor rose autogenously to a maximum between 800 and 1000 psi due to the factors described in Example 1. After cooling and relief of residual pressure, the light hydrocarbon fraction was isolated from the water, catalyst, and wax fractions as the supernatant following centrifugation of the product mixture; and the wax fraction was separated from the catalyst and water-soluble byproducts in the aqueous fraction by liquid-liquid extraction into hexane. The lower-MW hydrocarbon product corresponding to the combined liquid hydrocarbon and wax fractions was obtained in a yield of 65%, relative to the feedstock, and consisted of a mixture of substantially saturated straight-chain and methyloligopropylene alkanes, together with aromatics from PS, whose aggregated BN was 27±0.2 g bromine/100 g sample and whose distribution in carbon number subfractions was: ≤20, 59%; 21 to 30, 22%; 31 to 40, 9%; and >40, 10%.

### Example 7. Waste PE-PP in Continuous Flow

The process apparatus included a Parr 4580 series HT/HP stirred, closed tank reactor with a volume of 5 L configured with inlets for continuously receiving the feedstock with catalyst and water, a stirrer for agitating the mixture, and an outlet for the continuous removal of volatile, lower-MW compounds produced. The feedstock consisted of two waste plastics combined in a ratio of about 1:1 w/w, one containing about 95% PE whose melt index was 12 g/10 min (192° C./2.16 kg) and the other about 95% PP whose melt index was 13 g/10 min (230° C./2.16 kg). Prior to receiving the feedstock, catalyst, and water, the reactor was preconditioned by heating to the process temperature, 420° C., then charging with water by means of an HPLC pump coupled to a reactor inlet, where the amount of water was sufficient to achieve a pressure of 600 to 800 psi and where pressure was controlled by means of a back pressure regulator (BPR) coupled to port at the top of the reactor. Upon equilibration of temperature and pressure, the reactor received the feed through an inlet as a continuously-flowing extrudate from a Haake Polylab Rheomix 19/25 QC (Thermo Electron (Carlsruhe) GmbH), a single screw extruder configured to heat the feedstock to 325° C., to obtain a low-viscosity melt, and combine the same with catalyst in an amount of 2% w/w, relative to the feedstock, where the catalyst was bentonite clay. The HPLC pump continued to charge water into the reactor at a rate corresponding to 20% w/w, relative to the feed, and was dispersed in the same by operation of the stirrer at 200 rpm. Continued heating and stirring under a pressure controlled at 600 to 800 psi promoted (i) heat- and mass transfer and (ii) Initial Conversion and Main Conversion of PE and PP by reactions and mechanisms described for PE in Example 1 while the feedstock charge rate was controlled to provide an average residence time in the reactor of about 2 hours. After about 60 to 90 minutes, lower-MW product compounds that began accumulating in the headspace above the reaction mixture flowed through the BPR and were received into a first and a second condenser in series maintained at 70° C. and 5° C., respectively, while nonvolatile feedstock contaminants, reaction byproducts, and catalyst were removed through an outlet in the reactor bottom. The lower-MW hydrocarbon product was obtained in a yield of 82%, relative to the feedstock, and consisted of a mixture of substantially saturated straight-chain and methyloligopropylene alkanes, together with aromatics from PS, whose aggregated BN was 34±0.5 and whose distribution in carbon number subfractions was: ≤20, 68%; 21 to 30, 8%; 31 to 40, 5%; and >40, 19%.

### Example 8. Mixed Polyolefins in Waste Plastic, in Continuous Flow

The process apparatus included the same reactor used in Example 7. The feedstock whose plastic fraction contained PE, PP, PET, PS, and nylon in respective amounts of about 85%, 5%, 5%, 3%, and 2%; paper and inorganics were in amounts of about 2% relative to plastic. Prior to receiving the feedstock, catalyst, and water, the reactor was preconditioned by heating to the process temperature, 425° C., then charging with water by means of an HPLC pump coupled to a reactor inlet, where the amount of water was sufficient to achieve a pressure of 600 to 800 psi and where pressure was controlled by means of a back pressure regulator (BPR) coupled to port at the top of the reactor. Upon equilibration of temperature and pressure, the reactor received the feed through an inlet as a continuously-flowing extrudate from the same single screw extruder used in Example 7, which heated the feedstock to 325° C., to obtain a low-viscosity melt, and combined the same with catalyst in an amount of 2% w/w, relative to the feedstock, where the catalyst was bentonite clay. The HPLC pump continued to charge water into the reactor at a rate corresponding to 20% w/w, relative to the feed, and was dispersed in the same by operation of the stirrer at 200 rpm. Continued heating and stirring under a pressure controlled at 600 to 800 psi promoted (i) heat- and mass transfer and (ii) Initial Conversion and Main Conversion of PE, PP, and PS by reactions and mechanisms described for PE in Example 1 while the feedstock charge rate was controlled to provide an average residence time in the reactor of about 2 hours. After about 60 to 90 minutes, lower-MW product compounds that began accumulating in the headspace above the reaction mixture flowed through the BPR and were received into a first and a second condenser in series maintained at 70° C. and 5° C., respectively, while nonvolatile feedstock contaminants, reaction byproducts, and catalyst were removed through an outlet in the reactor bottom. The lower-MW hydrocarbon product was obtained in a yield of 85%, relative to the feedstock, and consisted of a mixture of substantially saturated straight-chain and methyloligopropylene alkanes, together with aromatics from PS, whose aggregated BN was 25±0.5 and whose distribution in carbon number subfractions was: ≤20, 65%; 21 to 30, 7%; 31 to 40, 3%; and >40, 25%.

### Example 9. Sequential Conversion of PE

Initial Conversion of PE. The process apparatus for Initial Conversion consisted of two ZE28BP twin-screw extruders (KraussMaffei) in series; the feedstock was waste plastic containing about 95% PE, whose average molecular weight was 280 kiloDaltons (kDa); and the catalyst was bentonite clay. Feedstock blended with 2.5% w/w catalyst was fed through the extruders at a rate of 3.0 kg/hour and average residence time of the feed in the extruders was about 8 minutes. The first extruder, whose barrel temperature was 300° C., melted the PE while the operation of the screws dispersed the catalyst in feedstock and promoted contacting between PE and catalyst to obtain an initial mixture as the first extrudate. That mixture was received by the second extruder, whose barrel temperature of 400° C., in combination with the operation of the screws, promoted efficient heat and mass transfer to obtain and an initial reaction mixture wherein catalyst-promoted bond scission in PE chains achieved Initial Conversion such that the second extrudate had water-like viscosity. The average molecular weight of the product from Initial Conversion was 70 kiloDaltons (kDa), which at one-fourth that of PE in the feedstock corresponds to an Extent of Conversion of 0.60.

Main Conversion. The process apparatus consisted of the same reactor used in Example 1 and the feed charged into it was the product obtained by Initial Conversion, which contained the bentonite clay catalyst in the amount of 2.5%. The amount of water charged into the reactor was 5% w/w, relative to the feed, and the total mixture occupied about 50% of the reactor volume. Under continuous stirring the product from Initial Conversion was heated to 420° C. to obtain a main reaction mixture and maintained there for a residence time of 2 hours to promote (i) heat- and mass transfer and (ii) Main Conversion by reactions and mechanisms described for PE in Example 1. At the process temperature, pressures in the reactor rose autogenously to a maximum between 1300 and 1500 psi due to the factors described in Example 1. After cooling and relief of residual pressure, the light hydrocarbon fraction was isolated from the water, catalyst, and wax fractions as the supernatant following centrifugation of the product mixture; and the wax fraction was separated from the catalyst and water-soluble byproducts in the aqueous fraction by liquid-liquid extraction into hexane. The lower-MW hydrocarbon product corresponding to the combined liquid hydrocarbon and wax fractions was obtained in a yield of 74%, relative to the feedstock, and consisted substantially of saturated straight-chain alkanes whose aggregated BN was 22±1 g bromine/100 g sample and whose distribution in carbon number subfractions was: ≤20, 50%; 21 to 30, 18%; 31 to 40, 11%; and >40, 21%.

### Example 10. Sequential Conversion of PP

Initial Conversion of PP. The process apparatus for Initial Conversion consisted of the same extruders configured as described in Example 9; the feedstock was waste plastic containing about 95% PP, whose average molecular weight was 320 kDa; and the catalyst was bentonite clay. Feedstock blended with 1.86% w/w catalyst was fed through the extruders at a rate of 3.0 kg/hour and average residence time of the feed in the extruders was about 8 minutes. The first extruder, whose barrel temperature was 300° C., melted the PP while the operation of the screws dispersed the catalyst in feedstock and promoted contacting between PP and catalyst to obtain an initial mixture as the first extrudate. That mixture was received by the second extruder, whose barrel temperature of 400° C., in combination with the operation of the screws, promoted efficient heat and mass transfer to obtain and an initial reaction mixture wherein catalyst-promoted bond scission in PP chains achieved Initial Conversion such that the second extrudate had water-like viscosity. The average molecular weight of the product from Initial Conversion was 28 kDa, which at slightly more than one-eleventh that of PP in the feedstock corresponds to an Extent of Conversion of about 1.06.

Main Conversion. The process apparatus consisted of the same reactor used in Example 1 and the feed charged into it was the product obtained by Initial Conversion, which contained the bentonite clay catalyst in the amount of 1.86%. The amount of water charged into the reactor was 10% w/w, relative to the feed, and the total mixture occupied about 25% of the reactor volume. Under continuous stirring the product from Initial Conversion was heated to 395° C. to obtain a main reaction mixture and maintained there for a residence time of 2 hours to promote (i) heat- and mass transfer and (ii) Main Conversion by reactions and mechanisms described for PE in Example 1. At the process temperature, pressures in the reactor rose autogenously to a maximum between 1200 and 1400 psi due to the factors described in Example 1. After cooling and relief of residual pressure, the light hydrocarbon fraction was isolated from the water, catalyst, and wax fractions as the supernatant following centrifugation of the product mixture; and the wax fraction was separated from the catalyst and water-soluble byproducts in the aqueous fraction by liquid-liquid extraction into hexane. The lower-MW hydrocarbon product corresponding to the combined liquid hydrocarbon and wax fractions was obtained in a yield of 90%, relative to the feedstock, and consisted substantially of methyloligopropylene alkanes whose aggregated BN was 58±1 g bromine/100 g sample and whose distribution in carbon number subfractions was about: ≤20, 78%; 21 to 30, 13%; 31 to 40, 4%; and >40, 5%.

Some portions of above description present the features of the present invention in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times, to refer to these arrangements of operations as modules or by functional names, without loss of generality. It should be noted that the process steps and instructions of the present invention could be embodied in software, firmware or hardware, and when embodied in software, could be downloaded to reside on and be operated from different platforms used by real time network operating systems. Moreover, the particular naming of the components, capitalization of terms, the attributes, data structures, or any other programming or structural aspect is not mandatory or significant, and the mechanisms that implement the invention or its features may have different names, formats, or protocols.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. It should be further understood that any of the features described with respect to one of the embodiments described herein may be similarly applied to any of the other embodiments described herein without departing from the scope of the present invention. As used in this document, “each” refers to each member of a set or each member of a subset of a set.

The present invention has been described in particular detail with respect to various possible embodiments, and those of skill in the art will appreciate that the invention may be practiced in other embodiments. First, the particular naming of the components, capitalization of terms, the attributes, data structures, or any other programming or structural aspect is not mandatory or significant, and the mechanisms that implement the invention or its features may have different names, formats, or protocols. Further, the system may be implemented via a combination of hardware and software, as described, or entirely in hardware elements. Also, the particular division of functionality between the various system components described herein is merely exemplary, and not mandatory; functions performed by a single system component may instead be performed by multiple components, and functions performed by multiple components may instead performed by a single component.

Various systems may also be used with programs in accordance with the teachings herein, or it may prove convenient to construct more specialized apparatus to perform the required method steps. The required structure for a variety of these systems will be apparent to those of skill in the art, along with equivalent variations.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

Finally, it should be noted that the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the following claims. It should be further understood that any of the features described with respect to one of the embodiments described herein may be similarly applied to any of the other embodiments described herein without departing from the scope of the present invention.

Having thus described the invention, what is claimed is:

1. A method for converting a feedstock comprising one or more polyaddition polymers (PAP) into lower-molecular-weight hydrocarbons, the method comprising:
(a) combining the feedstock with a first process agent (PA1) comprising one or more mineral-based catalysts to obtain an initial reaction mixture;
(b) heating the initial mixture to an initial conversion temperature T(init) in a range of about 390° C. to about 440° C. while dispersing PA1 to promote carbon-carbon bond scission in the PAP and to form reactive fragments F;
(c) in the presence of water in an amount sufficient to support in situ aqueous reforming (AR), generating hydrogen equivalents H(equiv) autogenously from at least a portion of the reactive fragments F;
(d) quenching at least a portion of other reactive fragments F with the H(equiv) to form stabilized fragments F-H;
(e) heating the initial mixture, with additional water as required, to a main conversion temperature T(main) in a range of about 390° C. to about 430° C. under a pressure of 200 to 1500 psi to promote iterative scission and quenching, thereby forming progressively smaller stabilized fragments F-H; and
(f) recovering from the reaction mixture a product mixture comprising lower-molecular-weight hydrocarbons that have an average carbon number of about C₆ to C₃₅ and are substantially saturated.

2. The method of claim 1, wherein the feedstock comprises one or more of polyethylene (PE), polypropylene (PP), polystyrene (PS), polyisobutylene (PIB), or tire rubber (TR).

3. The method of claim 2, wherein PA1 comprises one or more minerals selected from montmorillonite clay, bentonite clay, kaolinite clay, laterite clay, mordenite, hematite, hematite clay, and magnetite.

4. The method of claim 3, wherein PA1 is present at about 1 wt % to about 4 wt % relative to the feedstock.

5. The method of claim 1, wherein water present in the reaction mixture totals about 10 wt % to about 15 wt % relative to the feedstock, inclusive of water in the feedstock, water associated with PA1, and added water.

6. The method of claim 1, wherein the method is conducted at a pressure of about 200 psi to about 1500 psi generated autogenously by at least water vapor and gaseous products of aqueous reforming.

7. The method of claim 1, wherein no second process agent (PA2) hydrogen-source is added and the entirety of H(equiv) is generated autogenously by aqueous reforming of fragments F and optionally ethylene glycol derived from hydrolysis of polyester contaminants.

8. The method of claim 1, wherein autogenous AR proceeds according to:

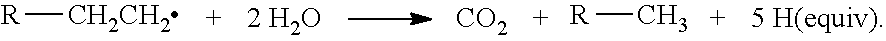

9. The method of claim 1, further comprising hydrolyzing contaminants comprising polyesters and/or polyamides in the feedstock using water present in the reaction mixture.

10. The method of claim 1, wherein the lower-molecular-weight hydrocarbons have a bromine number (BN) in a range of about 17 to about 41 g Br₂ per 100 g sample.

11. The method of claim 2, wherein the PAP includes polyethylene, and the lower-molecular-weight hydrocarbons comprise substantially straight-chain paraffins with single-digit weight percent unsaturation.

12. The method of claim 2, wherein the PAP includes polypropylene, and the lower-molecular-weight hydrocarbons comprise methyl-oligopropylene isoparaffins bearing a methyl group on every second backbone carbon.

13. The method of claim 2, wherein the PAP includes polystyrene, and the lower-molecular-weight hydrocarbons comprise mono-alkyl benzenes and polyphenyl alkanes that are substantially saturated apart from aromatic rings derived from polystyrene.

14. The method of claim 2, wherein the PAP includes tire rubber, and the method further comprises desulfurizing the feedstock by converting sulfur-containing linkages to hydrogen sulfide during conversion.

15. The method of claim 1, wherein T(init) is applied for about 0.1 h to about 1 h, and T(main) is applied for about 0.5 h to about 5 h.

16. The method of claim 1, wherein said (a)-(d) produces fragments F and F-H whose average size is about one-fourth to about one-fiftieth of the average size of the PAP in the feedstock.

17. The method of claim 1, operated so that scission-quenching rates for low-carbon-number products are self-limiting, thereby suppressing over-conversion to C₁-C₄ gases.

18. The method of claim 1, performed continuously and including continuously removing volatile lower-molecular-weight hydrocarbons from a reactor headspace while maintaining the reaction mixture at T(main) and a controlled pressure.

19. The method of claim 1, further comprising a multi-pass operation in which a distillation residue containing PA1 and heavier hydrocarbons is recycled to the conversion step for further scission-quenching.

20. (canceled)

21. The method of claim 1, wherein PA1 preparation includes one or more of washing, acid leaching and activation, and activation and/or impregnation with one or more metals or other elements.

22-25. (canceled)

26. The method of claim 1, further comprising preheating the feedstock to about 200° C. to about 370° C. to reduce viscosity and to obtain a melt before contacting with PA1.

27. (canceled)

28. The method of claim 1, wherein the overall liquid product yield is at least about 60 wt % in single-pass operation and at least about 80-90 wt % with multi-pass operation.

29. The method of claim 1, wherein the lower-molecular-weight hydrocarbons are naphtha-range feedstocks suitable for steam cracking to produce olefin monomers.

30-31. (canceled)

32. The method of claim 1, wherein:
said heating (b) further comprises in a first subsystem including one or more extruders, receiving the feedstock and PA1, melting the feedstock at about 200-370° C., and subsequently heating to T(init) for about 0.1-1 hour;
said heating (e) is effected in a second subsystem including a stirred tank reactor with pressure control and continuous water dosing; and
said recovering (f) is effected in a third subsystem including a distiller, recovering lower-molecular-weight hydrocarbons by distillation.

33-34. (canceled)

35. The method of claim 32, wherein the first subsystem comprises at least one of said one or more extruders configured to perform Initial Conversion to reduce MW of PE from about 280 kDa to about 70 kDa (Extent of Conversion≈0.60) before Main Conversion in the second subsystem at about 420° C. and about 5-10 wt % water.

36. (canceled)

37. The method of claim 1, wherein the conversion is carried out with about 10-20 wt % water relative to the feedstock and PA1 charge is reduced relative to polyethylene or polypropylene conversion due to inorganic additives in tire rubber imparting catalytic activity.

38. (canceled)

39. The method of claim 1, further comprising adding a second process agent (PA2) comprising CᵤHᵥO_{w} where 3≥u/w≥1, selected from methanol, ethanol, ethylene glycol, glycerol, or cellulose, to supplement H(equiv) generation.

40-46. (canceled)

47. A hydrocarbon composition produced by the method of claim 1, the composition comprising:
(a) a plurality of hydrocarbons having carbon numbers 6 to 35;
(b) being substantially saturated, with an aggregated bromine number of about 17 to 41 g Br₂/100 g;
(c) exhibiting minimal heteroatom incorporation, despite the presence of polymeric contaminants in the feedstock; and
(d) comprising structures substantially corresponding to the parent PAP, including one or more of: straight chain alkanes from PE, methyloligopropylene compounds (MOP) from PP, methyloligoisobutylene compounds (MOB) from PIB, and benzyl-derived hydrocarbons from PS.

48-50. (canceled)

51. The hydrocarbon composition of claim 47, wherein the feedstock comprises tire rubber, and the product is desulfurized relative to the feedstock and comprises paraffinic, isoparaffinic, and aromatic hydrocarbons.

52-53. (canceled)

54. A system for converting a feedstock comprising polyaddition polymers into lower-molecular-weight hydrocarbons, comprising:
(i) a feed handling unit configured to heat the feedstock to about 200-370° C.;
(ii) a PA1 delivery and dispersion unit configured to disperse a mineral-based catalyst into the heated feedstock;
(iii) an Initial Conversion reactor configured to maintain about 390-440° C. and provide mixing to promote PA1-mediated carbon-carbon scission;
(iv) a Main Conversion reactor configured to receive the output of the Initial Conversion reactor, disperse water, maintain about 390-430° C. at about 200-1500 psi, and facilitate autogenous generation of H(equiv) and quenching of reactive fragments; and
(v) a product recovery unit configured to separate lower-molecular-weight hydrocarbons.

55-59. (canceled)
